(19) United States
(12) Patent Application Publication
(10) Pub. No.: US 2014/0105960 A1
(43) Pub. Date: Apr. 17, 2014

(54) HYDROGELS FOR TISSUE REGENERATION

(71) Applicant: Massachusetts Institute of Technology, Cambridge, MA (US)

(72) Inventors: JANETA ZOLDAN, Cambridge, MA (US); Robert S. Langer, Newton, MA (US); Daniel S. Kohane, Newton, MA (US); Daniel Griffith Anderson, Sudbury, MA (US); Akihiko Kusanagi, Tokyo (JP); Hila Epstein-Barash, Newton, MA (US); Beata Chertok, Somerville, MA (US)

(73) Assignees: Children's Medical Center Corporation, Boston, MA (US); Massachusetts Institute of Technology, Cambridge, MA (US)

(21) Appl. No.: 14/050,320

(22) Filed: Oct. 9, 2013

Related U.S. Application Data
(60) Provisional application No. 61/713,462, filed on Oct. 12, 2012.

(51) Int. Cl. A61K 38/18; A61K 47/36; A61K 47/38; A61K 35/44

(52) U.S. Cl. CPC A61K 38/1866; A61K 35/44; A61K 38/1858; A61K 47/36; A61K 47/38
USPC 424/450; 424/93.7; 514/8.1; 514/8.2; 514/781; 514/777

(57) ABSTRACT

Provided herein are hydrogels and hydrogel-forming compositions that are useful for, among others, tissue regeneration in vivo. Methods for generating such hydrogels, for example, from such hydrogel-forming compositions are also provided herein. Therapeutic methods employing hydrogels and hydrogel-forming composition, for example, for restoration of tissue perfusion in the context of acute ischemia, are also provided. The disclosure also describes kits comprising components useful for generating hydrogels as described herein.

### RELATED APPLICATIONS

This application claims priority under 35 U.S.C. §119(e) to U.S. provisional patent application No. 61/713,462, filed Oct. 12, 2012, the entire contents of which are incorporated herein by reference.

### GOVERNMENT SUPPORT

This invention was made with U.S. Government support under grants DE-016516 and HL-060435 awarded by the National Institutes of Health. The U.S. Government has certain rights in this invention.

### BACKGROUND

Embryonic stem cells (“ES cells” or “ESCs”) as well as ES cell-derived progenitor cells represent promising cell sources for the regeneration of damaged or lost tissue. ES cells have the ability to differentiate into essentially any cell type of the body, proliferate indefinitely, and organize into complex multi-cell type tissue structures during embryonic-like differentiation. Progenitor cells are typically highly proliferative and are able to differentiate into cell types of a defined spectrum upon reception of appropriate molecular cues. Accordingly, ES and progenitor cells could, in theory, be used to rapidly replace or replenish endogenous, differentiated cells in damaged or injured tissues. However, stem or progenitor cell-based tissue regeneration approaches have been hampered by a lack of viable strategies to integrate progenitor cells and their offspring into injured tissue at the site of injury and also by difficulties to direct and control differentiation into desired cell types after administration.

### SUMMARY

Some aspects of this disclosure are based on the discovery that engineered hydrogels as provided herein can be used to retain injected stem or progenitor cells at or close to the site of injury in damaged tissue and also to efficiently direct differentiation of stem or progenitor cells in vivo based on the ability to control their exposure to molecular cues, such as one or more growth factors. Accordingly, such hydrogels are useful in stem- or progenitor cell-based approaches to restore function to lost or damaged tissue in vivo. Some aspects of this disclosure provide engineered hydrogels, hydrogel-forming compositions, methods for the manufacture of engineered hydrogels and for their use in vitro and in vivo, as well as kits comprising reagents and components for the generation of engineered hydrogels.

Some aspects of this invention provide engineered hydrogels. In some embodiments, the hydrogel comprises (a) a population of stem or progenitor cells that differentiate into a desired cell type in response to a growth factor; (b) the growth factor of (a) in a controlled-release form; and (c) a hydrogel scaffold encapsulating the cells of (a) and the controlled-release form of (b). In some embodiments, the hydrogel comprises a plurality of growth factors. In some embodiments, at least two growth factors are in different controlled-release forms. In some embodiments, the different controlled-release forms exhibit different release kinetics. In some embodiments, the different controlled-release forms exhibit different rates of release. In some embodiments, the controlled-release form is a liposome-encapsulated form. In some embodiments, the liposomes in which the growth factors are encapsulated are selected from the group consisting of DMPC liposomes (which have a high rate of release) and DSPC liposomes (which have a low rate of release). In some embodiments, the cells differentiate into cells that form blood vessels in response to the growth factor. In some embodiments, the population of stem or progenitor cells comprises endothelial progenitor cells. In some embodiments, the hydrogel comprises VEGF in a controlled-release form exhibiting a high rate of release and PDGF in a controlled-release form exhibiting a low rate of release. In some embodiments, the hydrogel scaffold comprises a polysaccharide. In some embodiments, the polysaccharide of the hydrogel scaffold is selected from the group consisting of carboxymethylcellulose (CMC), hyaluronic acid (HA), and dextran (DEX). In some embodiments, the polysaccharide molecules of the hydrogel are covalently bound to each other via hydrazone bonds. In some embodiments, the average pore size of the hydrogel is smaller than the average diameter of the cells of (a) and/or than the average diameter of the controlled-release form of (b).

Some aspects of this disclosure provide hydrogel-forming compositions. In some embodiments, the composition comprises (a) a growth factor in a controlled-release form; (b) a polymer comprising a first reactive moiety; and (c) a polymer comprising a second reactive moiety that forms a covalent bond with the first reactive moiety under physiological conditions, thus forming a hydrogel. In some embodiments, the composition comprises (d) a population of stem or progenitor cells that differentiates into a desired cell type in response to the growth factor of (a). In some embodiments, the composition comprises a plurality of growth factors. In some embodiments, at least two growth factors are in different controlled-release forms. In some embodiments, the different controlled-release forms exhibit different release kinetics. In some embodiments, the controlled-release form is a liposome-encapsulated form. In some embodiments, the liposomes in which the growth factors are encapsulated are selected from the group consisting of DMPC liposomes (high rate of release) and DSPC liposomes (low rate of release). In some embodiments, the cells of (d) differentiate into cells that form blood vessels in response to the growth factor. In some embodiments, the population of cells comprises endothelial progenitor cells. In some embodiments, the hydrogel comprises VEGF in a controlled-release form exhibiting a high rate of release and PDGF in a controlled-release form exhibiting a low rate of release. In some embodiments, the polymer of (b) and the polymer of (c) are provided in separate aqueous solutions for administration to a subject (e.g., injection or implantation). In some embodiments, the cells of (d) and the growth factor of (a) are suspended in one of the aqueous solutions, either together or separately. In some embodiments, the separate aqueous solutions are combined before or upon administration to a subject, e.g., injection or implantation, and combining the solutions results in covalent crosslinking of the polymer of (b) with the polymer of (c). In some embodiments, the polymer of (b) and/or the polymer of (c) comprises or consists of a polysaccharide. In some embodiments, the polymer of (b) and/or the polymer of (c) are, individually and independently, selected from the group consisting of carboxymethylcellulose (CMC), hyaluronic acid (HA), and dextran (DEX). In some embodiments, the reactive moieties are click chemistry moieties. In some embodiments, the first reactive moiety is an aldehyde moiety, the second reactive moiety is an adipic anhydride moiety, and the covalent bond is a hydrazone bond. In some embodiments, the composition comprises a multi-compartment syringe comprising the polymer of (b) and the polymer of (c) in different compartments, and a nozzle for mixing the polymers. In some embodiments, the polymers may, individually and independently, be polysaccharides. In some embodiments, the polymer of (b) and the polymer of (c) are different polymers or derived from different polymers, e.g., in some embodiments, the polymer of (b) may be DEX and the polymer of (c) may be CMC. In other embodiments, the polymers of (b) and (c) are the same or derived from the same polymer. For example, in some embodiments, the polymer of (b) may be CMC functionalized with an aldehyde reactive moiety and the polymer of (c) may be CMC functionalized with an adipic anhydride reactive moiety.

Some aspects of this disclosure provide therapeutic methods comprising administering a hydrogel or a hydrogel-forming composition described herein to a subject in need thereof. In some embodiments, the subject is a subject in need of tissue regeneration. In some embodiments, the hydrogel or hydrogel-forming composition comprises a growth factor and a population of cells capable of regenerating the tissue in the presence of the growth factor. In some embodiments, the subject is a subject in need of revascularization of a tissue. In some such embodiments, the hydrogel or the hydrogel-forming composition comprises endothelial progenitor cells, VEGF in a controlled-release form exhibiting a high rate of release, and PDGF in a controlled-release form exhibiting a low rate of release.

Some aspects of this disclosure provide methods for generating a hydrogel. In some embodiments, the method comprises providing (a) a growth factor in a controlled-release form; (b) a polymer comprising a first reactive moiety; and (c) a polymer comprising a second reactive moiety that forms a covalent bond with the first reactive moiety under physiological conditions; and contacting the polymer of (b) with the polymer of (c) in the presence of the controlled-release form of the growth factor of (a), thus forming a hydrogel encapsulating the controlled-release form of the controlled-release form of the growth factor of (a). In some embodiments, the method further comprises providing (d) a population of stem or progenitor cells that differentiates into a desired cell type in response to the growth factor of (a). In some embodiments, the polymer of (b) is contacted with the polymer of (c) in the presence of the controlled-release form of the growth factor of (a) and the cells of (d), thus forming a hydrogel encapsulating the controlled-release form of the growth factor of (a) and the cells of (d). In some embodiments, the growth factor of (a) comprises a plurality of growth factors. In some embodiments, at least two growth factors are in different controlled-release forms. In some embodiments, the different controlled-release forms exhibit different release kinetics. In some embodiments, the controlled-release form is a liposome-encapsulated form. In some embodiments, the liposomes in which the growth factors are encapsulated are selected from the group consisting of DMPC liposomes (high rate of release) and DSPC liposomes (low rate of release). In some embodiments, the cells of (d) differentiate into cells that form blood vessels in response to the growth factor. In some embodiments, the population of cells comprises endothelial progenitor cells. In some embodiments, the hydrogel comprises VEGF in a controlled-release form exhibiting a high rate of release and PDGF in a controlled-release form exhibiting a low rate of release. In some embodiments, the polymer of (b) and the polymer of (c) are provided in separate aqueous solutions for injection. In some embodiments, the cells of (d) and the controlled-release form of the growth factor of (a) are suspended in one of the aqueous solutions, either together or separately. In some embodiments, the polymers may, individually and independently, be polysaccharides. In some embodiments, the polymer of (b) and/or the polymer of (c) comprises or consists of a polysaccharide. In some embodiments, the polymer of (b) and/or the polymer of (c) are, individually and independently, selected from the group consisting of carboxymethylcellulose (CMC), hyaluronic acid (HA), and dextran (DEX). In some embodiments, the polymer of (b) and the polymer of (c) are different polymers or derived from different polymers, e.g., in some embodiments, the polymer of (b) may be DEX and the polymer of (c) may be CMC. In other embodiments, the polymers of (b) and (c) are the same or derived from the same polymer. For example, in some embodiments, the polymer of (b) may be CMC functionalized with an aldehyde reactive moiety and the polymer of (c) may be CMC functionalized with an adipic anhydride reactive moiety. In some embodiments, the reactive moieties are click chemistry moieties. In some embodiments, the first reactive moiety is an aldehyde moiety, the second reactive moiety is an adipic anhydride moiety, and the covalent bond is a hydrazone bond. In some embodiments, the method comprises administering the controlled-release form of the growth factor of (a), the polymer of (b), the polymer of (c), and, optionally, the cells of (d), to a subject. In some embodiments, the polymer of (b) is contacted with the polymer of (c) in the presence of the controlled-release form of the growth factor of (a) and, optionally, the cells of (d), upon administration or after administration in situ, thus forming a hydrogel encapsulating the controlled-release form of the growth factor of (a) and, optionally, the cells of (d), at the site of administration. In some embodiments, the method comprises combining the controlled-release form of the growth factor of (a), the polymer of (b), the polymer of (c), and, optionally, the cells of (d), and administering the combination to a subject under conditions suitable for the formation of a hydrogel encapsulating the controlled-release form of the growth factor of (a) and, optionally, the cells of (d), at the site of administration. In some embodiments, the subject is in need of regeneration of a tissue and the cells of (d) differentiate into a cell type regenerating the tissue in response to the growth factor of (a). In some embodiments, the subject is in need of restoration of blood flow to a tissue, the cells of (d) comprise endothelial progenitor cells, and the growth factor of (a) comprises VEGF in a release form having a high rate of release and PDGF in a release form having a low rate of release.

Some aspects of this disclosure provide kits. In some embodiments, the kit comprises (a) a polymer comprising a first reactive moiety; and (b) a polymer comprising a second reactive moiety, wherein the second reactive moiety forms a covalent bond with the first reactive moiety under physiological conditions, thus forming a hydrogel comprising the polymer of (a) covalently bound to the polymer of (b). In some embodiments, the kit further comprises (c) a growth factor in a controlled-release form. In some embodiments, the kit further comprises (d) a population of cells that differentiate into a desired cell type in response to the growth factor of (c). In some embodiments, the kit further comprises (e) an applicator, for example, an applicator that comprises a compartment for an aqueous solution comprising the polymer of (a); a compartment for an aqueous solution comprising the polymer of (b); and a mixing nozzle for mixing and/or administering the aqueous solutions. In some embodiments, the kit comprises a plurality of growth factors in different controlled-release forms, and wherein the different controlled-release forms exhibit different release kinetics. In some embodiments, the controlled-release form is a liposome-encapsulated form. In some embodiments, the liposomes in which the growth factors are encapsulated are selected from the group consisting of DMPC liposomes (high rate of release) and DSPC liposomes (low rate of release). In some embodiments, the kit comprises VEGF in a controlled-release form exhibiting a high rate of release and PDGF in a controlled-release form exhibiting a low rate of release. In some embodiments, the kit comprises a population of endothelial progenitor cells. In some embodiments, the polymers may, individually and independently, be polysaccharides. In some embodiments, the polymer of (a) and the polymer of (b) are different polymers or derived from different polymers. In other embodiments, the polymers of (a) and (b) are the same or derived from the same polymer.

Other advantages, features, and uses of the invention will be apparent from the detailed description, the drawings, and the claims.

### BRIEF DESCRIPTION OF THE DRAWINGS

FIG. 1. Exemplary hydrogel chemistries. (A) Functionalization of hyaluronic acid with reactive moieties (CHO: aldehyde, ADH: adipic dihydrazide). The functionalized polymers (HA-CHO and HA-ADH) react to form a hydrogel. (B) Hydrazone bond formation between different polysaccharides comprising an aldehyde (CHO) reactive moiety (HA: hyaluronic acid, CMC: Carboxymethylcellulose, DEX: dextran) and carboxymethylcellulose comprising an ADH reactive moiety (CMC-ADH).

FIG. 2. Exemplary liposomes for encapsulation and controlled release of growth factors. The upper panel shows a unilamellar vesicle (UV, left), with exemplary sites of encapsulation of three drugs of different hydrophilicity, and a multilamellar vesicle (MLV, right), having multiple alternate aqueous and lipid layers. Transmission electron micrographs show the size distribution in exemplary liposome fractions. DSPC: 1,2-distearoyl-sn-glycero-3-phosphocholine; DMPC: 1,2-dimyristoyl-sn-glycero-3-phosphocholine; Tm: melting temperature in ° C.

FIG. 3. Release kinetics of growth factors in hydrogels with and without controlled-release formulations. (A) Release kinetics of VEGF from different in situ formed hydrogels, controlled-release forms (DMPC or DSPC liposomes), and controlled-release forms embedded in hydrogels. (B) Release kinetics of PDGF from in situ formed DEX-CMC hydrogel, controlled-release forms (DMPC or DSPC liposomes), and controlled-release forms embedded in DEX-CMC hydrogels. (C) Controlled release of growth factors from DSPC and DMPC liposomes embedded in DEX-CMC hydrogel, showing different rates of release of PDGF from DSPC liposomes as compared to VEGF from DMPC liposomes.

FIG. 4. Microstructure of human embryonic stem cell-(hESC)-laden in situ cross linked hydrogels. Scanning electron microscope micrographs of in situ cross-linked hydrogels with (lower panel) and without hESC (upper panel). Porous microstructure of cross-linked hydrogels allows hESC growth inside these pores. Bright light and fluorescent micrographs of hESC laden in situ cross linked hydrogels showed that hESC, EB, and dissociated cells all exhibit high viability as measured by live/dead assay (not shown).

FIG. 5. Human ES cell-derived CD34⁺ cells grown in hydrogels for 2 weeks form vascular networks.

FIG. 6. Schematic of an exemplary therapeutic embodiment of a hydrogel-forming composition comprising two growth factors (VEGF and PDGF) provided in different controlled-release forms (DSPC liposomes—low rate of release, and DMPC liposomes—high rate of release), and co-entrapped with endothelial progenitor cells in a CMC-DEX hydrogel formed by contacting DEX-CHO with CMC-ADH. Upper panel shows a disrupted blood vessel to which the hydrogel-forming composition is administered (left), the formation of the hydrogel around the blood vessel at the site of administration after about 30 s (middle), and the formation of blood vessels bypassing the disrupted site after 6 weeks (right). The middle panel shows a schematic of gel and blood vessel formation over time, and the lower panel shows the respective hydrogel chemistry.

FIG. 7. Mouse hindlimb ischemia model before surgery, during induction of ischemia, during application of in situ-forming hydrogel comprising endothelial progenitor cells and growth factors, and after surgery.

FIG. 8. In vivo restoration of blood vessel function after hydrogel administration in mouse hindlimb ischemia model. Upper panel: visual examination of mice subjected to surgery revealed that control groups (left, “no Rx”) exhibited hind limb necrosis and amputation within 4-6 days, while mice treated with hydrogels comprising CD34 positive human ESC-derived cells and both VEGF and PDGF in controlled release liposomes (Lipo-GF) resulted in only minor necrosis and no amputation.

FIG. 9. Histological analysis of muscle bed in ischemic muscle with no treatment (no Rx) and ischemic muscle treated with hydrogels comprising CD34 positive hESC-derived cells as well as VEGF and PDGF in controlled-release liposomes (with Rx). General morphology was detected by hematoxylin and eosin (H&E) staining, while Trichrome staining detected muscle necrosis (muscle fibers stained blue instead of red).

FIG. 10. Analysis of hydrogel-mediated neovascularization in ischemic hind limb mouse model. A) micrographs of tissue sections immunohystochemically stained for endothelial (CD31) and fibroblast (SMA) cell markers. B) quantification of CD31 and SMA positive blood vessel size and total densities. (*** indicates p=0.05)

FIG. 11. Perfusion-reflecting contrast ultrasound scans of treated and untreated ischemic hind limbs as well as healthy hind limbs of typical experimental animals. (A) Visualization of perfusion by pseudo-color scale images of peak tissue contrast enhancement overlaid on the grayscale anatomical scans control and ischemic limb in mice that did not receive hydrogel treatment and mice that received treatment with hydrogels comprising CD34 positive-hESC derived cells and both VEGF and PDGF in controlled release liposomes. Untreated mice show normal blood flow in the control limb, while the ischemic limb shows almost no blood flow. In contrast, treated mice show blood flow in both limbs at comparable levels. (B) Axial scans of the right and the left hind limbs were acquired perpendicular to the lines marked. (C) Significant differences (*) in mean contrast pixel density within the cross-section rectangle of interest (ROI) in control and ischemic limbs from a number of treated and untreated mice.

FIG. 12. Immunostaining of cell populations in hydrogels in vivo. Expression of human endothelial markers was constrained within hydrogels or in their vicinity in the 6 weeks treated mice. Hydrogels contained vascular structures that stained positively for human CD31, human αSMA, human Von Willebrand factor (VWF) and that bound Ulex Europaeus Agglutinin I (UEA-1), a marker for human endothelial cells. Rhodamine or fluorescein conjugate secondary antibodies were used for fluorescent visualization of cells expressing human endothelial markers, followed by DAPI (4,6-diamidino-2-phenylindole) nuclear staining.

### DETAILED DESCRIPTION

Some aspects of this disclosure relate to the discovery that administration of engineered hydrogels, or hydrogel-forming compositions, comprising growth factors and stem or progenitor cells to an injured or dysfunctional tissue can be used to efficiently restore tissue function in vivo. Some aspects of this disclosure are based on the findings of an evaluation of the engineered hydrogels or hydrogel-forming compositions for control of stem and progenitor cell differentiation in vivo, e.g., in the context of therapeutic tissue regeneration approaches. Some aspects of this disclosure are based on the surprising discovery that engineered hydrogels and hydrogel-forming compositions as provided herein can deliver stem or progenitor cells able to differentiate into a desired cell type to an injured or dysfunctional tissue, and that such hydrogels retain cells at the site of administration, but do not interfere with their proliferation and differentiation, nor with tissue regeneration. Some aspects of this disclosure relate to the discovery that co-entrapment of stem or progenitor cells with controlled-release forms of growth factors that can direct stem or progenitor cell differentiation, in a hydrogel as provided herein, creates a synergistic effect by providing localized, controllable release of the growth factors to direct differentiation of gel-embedded cells retained at a site of injury or tissue dysfunction. Some aspects of the disclosure relate to the discovery that co-entrapment of controlled-release forms of growth factors and growth factor-responsive stem or progenitor cells, within a hydrogel as provided herein, can be used to stimulate differentiation processes requiring complex growth factor signaling patterns in vivo, such as sequential signaling of two or more growth factors. The engineered hydrogels provided herein, as well as the associated hydrogel-forming compositions and methods of synthesis, allow rapid restoration of tissue function, which can be used to treat acute clinical presentations, as demonstrated herein in an exemplary model of acute hind limb ischemia.

### Engineered Hydrogels and Hydrogel Forming Compositions

Some aspects of this invention provide engineered hydrogels. In some aspects, hydrogels provided herein are useful for delivery of stem or progenitor cells to a dysfunctional tissue, such as, for example, to a site of injury in a tissue that causes loss of tissue function, in order to regenerate the tissue, e.g., to restore or improve tissue function. The engineered hydrogels provided herein address several problems faced by cell-based approaches for tissue regeneration. The first problem is that stem or progenitor cells administered to a dysfunctional tissue or to a site of injury for tissue regeneration are typically not efficiently retained at the site of administration. Rather, such cells that are administered in a non-encapsulated manner, may be washed away by body fluid circulation, or migrate out of the site of injury. As a result, the stem or progenitor cells available for differentiation and tissue regeneration at the site of injury typically represent only a small fraction of the cells that were administered, and, in some cases, the amount of cells retained is insufficient to support any measurable integration into or regeneration of the dysfunctional tissue. The engineered hydrogels provided herein efficiently retain stem or progenitor cells at the site of administration, but do not interfere with the differentiation or proliferation of the administered cells nor with their capability to interact with the surrounding tissue and regenerate tissue function.

The term “tissue regeneration,” as used herein, refers to the restoration, full or in part, of a structure or a function of a tissue that exhibits a loss or impairment of that structure or function, for example, as a consequence of a disease or injury. The restoration of blood flow to an ischemic, hypoxic, or anoxic tissue, the restoration of the mechanical function of a broken bone, the restoration of neural function to a brain or spinal cord region after traumatic injury, or the restoration of glucose-responsive insulin production to pancreatic tissue of a type I diabetic are non-limiting examples of tissue regeneration. Additional examples will be apparent to those of skill in the art and the disclosure is not limited in this respect.

The term “hydrogel,” as used herein, refers to a gel in which water is the dispersion medium. Typically, a hydrogel comprises a plurality of polymer molecules that are cross-linked, either via covalent bonds or via non-covalent interactions, thus forming a polymer scaffold, also referred to herein as a hydrogel scaffold. In some preferred embodiments, the cross-linking is via covalent bonds. Cross-linking typically comprises inter-polymer bonds (bonds between different polymer molecules), but may also comprise intra-polymer bonds (bonds within the same polymer molecule). In some embodiments, the polymers are water-soluble in their non-cross-linked form, but are insoluble once they are cross-linked. A hydrogel scaffold is typically super-absorbent, and a hydrogel can comprise more than 99% water. Hydrogels useful in the context of this disclosure typically comprise a water content within the range of about 85% to about 99%. For example, in some embodiments, a hydrogel provided herein comprises a water content of about 99%, about 98%, about 97.5%, about 97%, about 96%, about 94%, about 93%, about 92%, about 91%, or about 90%. In some embodiments, hydrogels with a water content of less than 90% are employed. A hydrogel may comprise components in addition to the scaffold and water, for example, cells, and/or drugs or compounds, e.g., growth factors in controlled-release form.

The term “hydrogel scaffold,” as used herein, refers to a water-insoluble network of polymers within a hydrogel.

The term “polymer,” as used herein, refers to a molecule comprising a plurality of repeating structural units (monomers), typically at least 3, linked together via covalent bonds. Non-limiting examples of polymers are polysaccharides, polynucleotides, and polypeptides. Exemplary hydrogel-forming polymers, e.g., DEX, CMC, and HA, are described in more detail elsewhere herein. Additional polymers that can form hydrogels are also encompassed. In embodiments, where a hydrogel or hydrogel-forming composition is administered to a subject, the polymer and the respective hydrogel scaffold formed are preferably biocompatible in that they do not elicit an immune or inflammatory response once administered and in that the formation of the hydrogel scaffold does not result in toxic or otherwise harmful side reactions or side products.

In some embodiments, the polymers comprised in a hydrogel scaffold as provided herein are polysaccharides. The term “polysaccharide,” as used herein, refers to a polymer of sugars, which are also often referred to as monosaccharides. Most polysaccharides are aldehydes or ketones, typically comprising one hydroxyl group per carbon atom of the molecule, and, thus, many polysaccharides are of the molecular formula CₙH₂ₙOₙ. However, polysaccharides that do not conform to this generic formula are also known to those of skill in the art and may be included in the hydrogels or hydrogel-forming compositions provided herein. In some embodiments, a polysaccharide includes 3 or more, 4 or more, 5 or more, or 6 or more sugar monomers or monosaccharide units. Exemplary polysaccharides that can form hydrogel scaffolds include, without limitation, dextrans, cellulose derivatives, hyaluronic acid, starch derivatives, and glycogen. In some embodiments, the polysaccharides of the hydrogel are covalently bound to each other via hydrazone bonds. In some embodiments, the polysaccharide molecules of the hydrogel are bound via non-covalent interactions, e.g., as is the case with alginate hydrogels, via chelation of a divalent cation such as Mg²⁺, Ca²⁺, Sr²⁺, or Ba²⁺. The bonds in the hydrogel can be intra-polysaccharide bonds or inter-polysaccharide bonds.

In some embodiments, engineered hydrogels are provided that comprise cross-linked dextran (DEX), hyaluronic acid (HA), or carboxymethylcellulose (CMC), either individually or in any combination.

The term “carboxymethylcellulose” or “CMC,” as used herein, refers to a cellulose derivative with carboxymethyl groups (—CH₂—COOH) bound to some of the hydroxyl groups of the glucopyranose monomers that make up the cellulose backbone. An exemplary structure of a CMC polymer is shown in the following formula:

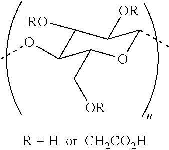

Those of skill in the art will understand that the disclosure is not limited to this exemplary structure.

The term “dextran” or “DEX,” as used herein, refers to a complex, branched glucan (a polymer of glucose monomers) composed of chains of varying lengths (from 3 to 2000 kilodaltons). An exemplary structure of a DEX polymer is shown in the following formula:

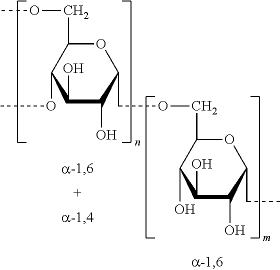

Those of skill in the art will understand that the disclosure is not limited to this exemplary structure.

The term “hyaluronic acid” or “HA,” as used herein, refers to an anionic, nonsulfated glycosaminoglycan. An exemplary structure of an HA polymer is shown in the following formula:

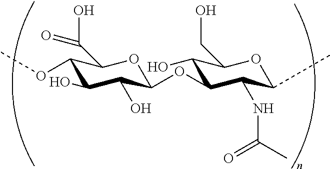

Those of skill in the art will understand that the disclosure is not limited to this exemplary structure.

It will be apparent to the skilled artisan that any suitable hydrogel scaffold can be employed in some embodiments of this disclosure, and that the exemplary scaffolds and hydrogel-forming polymers described herein in more detail are not in any way limiting. For example, in some embodiments, engineered hydrogels are provided that comprise polymer scaffolds made of polymers that are known in the art to be useful in the preparation of hydrogels. Such polymers may include, in some embodiments, e.g., cellulose derivatives, xyloglucans, chitosans, glycerophosphates, alginates, gelatin, polyethylene glycol, N-isopropylamide copolymers (e.g., poly(N-isopropylacrylamide-co-acrylic acid) or poly(N-isopropylacrylamide)/poly(ethylene oxide)), poloxamers (e.g., pluronic-modified poloxamer or poloxamer/poly(acrylicacid)), poly(ethylene oxide)/poly(D,L-lactic acid-co-glycolic acid), poly(organophosphazene), or poly(1,2-propylene phosphate), and their derivatives. Additional polymers useful for the formation of a hydrogel scaffold in the context of some embodiments of this disclosure will be apparent to those of skill in the art, and the disclosure is not limited in this respect.

The hydrogels provided herein typically comprise hydrogel scaffolds made of cross-linked polymers. The term “cross-linked,” as used herein, refers to a type of binding involving a plurality of polymers and a plurality of binding interactions. Cross-linked polymers are polymers that are connected to form a network, and, in the context of hydrogels, a hydrogel scaffold. Accordingly, a polymer in a cross-linked state is connected to another polymer or a plurality of other polymers through two or more covalent bonds or non-covalent interactions, thus forming a network of interconnected polymer molecules. Cross-linking can be either via covalent bonds or via non-covalent interactions. In some embodiments, hydrogel-forming polysaccharides cross-link via non-covalent bonds, e.g., as is the case for alginates, via chelation of ions. In other embodiments, however, the polymers forming the hydrogel scaffold of an engineered hydrogel provided herein are cross-linked via covalent bonds. The formation of such covalently cross-linked hydrogel scaffolds typically involves the formation of covalent bonds between individual polymer molecules, but may also involve the formation of intra-molecular bonds within the same polymer molecule.

In some embodiments, covalent bond-formation between hydrogel-forming polymer molecules involves a chemical reaction between reactive moieties comprised in or conjugated to the hydrogel-forming polymers. The term “reactive moiety,” as used herein in the context of hydrogel-forming polymers, refers to a moiety comprised in or conjugated to a first polymer that can react with a second reactive moiety comprised in or conjugated to a second polymer to form a covalent bond. In some embodiments, a hydrogel-forming polymer comprises or is conjugated to a plurality of reactive moieties, which allows for the generation of covalent cross-links with a number of polymer molecules. In some embodiments the plurality of reactive moieties comprised in or conjugated to a polymer are of the same type. In other embodiments, a polymer may comprise or be conjugated to a plurality of different reactive moieties.

In some embodiments, as for example in embodiments that relate to the in situ formation of a hydrogel in a dysfunctional tissue of a subject, preferred reactive moieties form a covalent bond under physiological conditions and do not produce any toxic side products when forming a covalent bond.

The term “physiological conditions,” as used herein, refers to a range of chemical (e.g., pH, ionic strength), biochemical (e.g., enzyme concentrations), and physical (e.g., temperature, pressure) conditions that can be encountered in intracellular and extracellular fluids of tissues, such as, for example, in the intracellular and extracellular fluids of a subject. For most cells and tissues, the physiological pH ranges from about 7.0 to about 7.5, the physiological ionic strength ranges from about 50 mM to about 400 mM, the physiological temperature ranges from about 20° C. to about 42° C., and the physiological pressure ranges from about 925 mbar to about 1050 mbar.

Suitable chemistries for in situ hydrogel formation include, without limitation, boronate esterification (e.g., phenylboronate-salicylhydroxamate conjugation), click chemistry reactions (e.g., 1,3-dipolar cycloaddition), Diels-Alder reactions, amidation via modified Staudinger ligation, as well as chemistries yielding imine, oxime, and hydrazone linkages. In some embodiments the reactive moiety is an anhydride, such as, for example, and adipic anhydride, and the second reactive moiety is an aldehyde moiety, and these moieties react to form a hydrazone bond. In some such embodiments, the reactive moiety of a first polymer forms a covalent bond with a reactive moiety of a second polymer, thus linking the polymers. In some embodiments, a single polymer partaking in such a reaction is conjugated to or comprises a plurality of reactive moieties of the same type, thus allowing cross-linking of the reactant polymers. In the context of hydrogel formation, suitable reactive moieties are typically stable in water and in air, comprise nontoxic functional groups that react without toxic side products, and the bond-forming reaction kinetics are rapid or controllable.

In some embodiments, the reactive moiety is a click chemistry moiety. The term “click chemistry,” as used herein, refers to a chemical philosophy introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate non-toxic byproducts, are stereospecific, exhibit a large thermodynamic driving force >84 kJ/mol to favor a reaction with a single reaction product, and/or can be carried out under physiological conditions. In particular, click chemistry reactions that can be carried out under physiological conditions and that do not produce toxic or otherwise harmful side products are suitable for the generation of hydrogels in situ. Reactive moieties that can partake in a click chemistry reaction are well known to those of skill in the art, and include, but are not limited to alkyne and azide, alkene and tetrazole or tetrazine, or diene and dithioester. Other suitable reactive click chemistry moieties suitable for use in the context of polymer functionalization for hydrogel generation are known to those of skill in the art.

The engineered hydrogels provided herein are typically porous structures, and the size and uniformity of the pores in the hydrogels as provided herein depends on, among other factors, the nature of the scaffold forming the structural basis of a given hydrogel, e.g., the composition of polymers forming the scaffold, the grade of cross-linking of polymers within the scaffold, and the concentration of the scaffold-forming polymers in the hydrogel, with higher densities typically associated with smaller pore size and vice versa. The size of the pores of a hydrogel determines, in turn, how well a hydrogel can retain a given molecule, cell, particle, or controlled-release form. The term “pore size,” as used herein in the context of hydrogels, refers to the diameter of the pores in a hydrogel scaffold. In some embodiments, the pore size is the average inner diameter of pores in a hydrogel. In other embodiments, the term refers to the smallest or the largest inner diameter of a pore found in a given hydrogel. The pore size of some hydrogels are known to those of skill in the art, and the pore size of a hydrogel in question can be determined with no more than routine experimentation, e.g., by subjecting the hydrogel to an imaging assay of suitable resolution, e.g., a scanning microscopy assay, as described herein, or to a size exclusion assay with a series of molecules of known molecular weight and/or diameter. In some embodiments, the pore size of a hydrogel used in the context of this disclosure is about 10 μm-about 100 μm, about 50 μm-about 250 μm, about 250 μm-about 500 μm, about 300 μm-about 700 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 1000 μm. Pore sizes that are larger or smaller than the ones enumerated immediately above may be used in some embodiments. The disclosure is not limited in this respect.

In some embodiments, the average pore size of the hydrogels is smaller than the average size of an agent, e.g., a growth factor in a controlled-release form, and/or cell encapsulated in the hydrogel. For example, if the hydrogel comprises a growth factor in a liposome-encapsulated form, with the average liposome diameter being about 200-300 μm, then, in some embodiments, the average pore size of the hydrogel is less than 200-300 μm, including, for example, 100-200 μm. Choosing an average pore size smaller than the average diameter of an agent to be encapsulated, here the controlled-release form of a growth factor, ensures that the agent is effectively retained by the hydrogel scaffold and cannot easily diffuse or otherwise leak out of the hydrogel scaffold. In some embodiments, the cells to be encapsulated within the hydrogel scaffold are smaller in diameter than the average pore size of the hydrogel, which, in turn, is smaller than the average diameter of the controlled-release form of the respective growth factor to be encapsulated. In some embodiments, cell adhesion, rather than hydrogel pore size, retains cells encapsulated in the gel within the hydrogel scaffold.

In some embodiments, engineered hydrogels are provided herein that comprise, embedded in the hydrogel scaffold, a population of cells, for example, a population of stem or progenitor cells. In some embodiments, hydrogel-forming compositions are provided herein that can form hydrogels comprising, embedded in the hydrogel scaffold, a population of cells, for example, a population of stem or progenitor cells.

The term “stem cell” as used herein, refers to a cell that is capable of dividing, of differentiating into diverse, specialized cell types, termed “differentiated cells,” and of self-renewal, which refers to a division that produces at least one daughter cell that itself is a stem cell. Exemplary stem cell types suitable for embedding into hydrogels according to some aspects of this disclosure include, without limitation, embryonic stem cells, fetal stem cells (including, e.g., umbilical cord stem cells), or adult stem cells (e.g., mesenchymal stem cells, endothelial stem cells, neuronal stem cells, adipose-derived stem cells, hematopoietic stem cells, or dental pulp stem cells). Biomarkers and methods for the identification, isolation, and culture of stem cells are known to those of skill in the art and it will be understood that the disclosure is not limited in this respect.

In some embodiments, the stem or progenitor cells comprised in the engineered hydrogels or hydrogel-forming compositions provided herein are, or are derived from embryonic stem cells, for example, from human embryonic stem cells. In some embodiments, the stem or progenitor cells comprised in the engineered hydrogels or hydrogel-forming compositions provided herein are, or are derived from adult stem cells, for example, from human neuronal, hematopoietic, bone marrow, bone, liver, skin, intestinal, endothelial, or pancreatic stem cells. In some embodiments, the stem or progenitor cells comprised in the engineered hydrogels or hydrogel-forming compositions provided herein are, or are derived from induced pluripotent stem cells (iPS cells), for example, iPS cells derived from a subject having a disease or disorder. In some embodiments, the use of iPS cells as a source of the stem or progenitor cells comprised in the engineered hydrogels or hydrogel-forming compositions allows for tissue regeneration with cells originating from the same subject that the hydrogel or hydrogel-forming composition is administered to.

The term “progenitor cell,” as used herein, refers to a cell that is capable of dividing, of differentiating into a specialized cell type or into a plurality of such cell types, but not of self-renewal. Progenitor cells are typically more differentiated than stem cells of the same tissue or developmental lineage, and are often an early product of stem cell division and differentiation. Some progenitor cells can divide for a limited number of times and subsequently lose their proliferative potential. Exemplary progenitor cell types suitable for embedding into hydrogels according to some aspects of this disclosure include, without limitation, satellite cells, e.g., from muscle tissue, intermediate progenitor cells of the subventricular zone, neural progenitor cells, bone marrow stromal cells, basal cells of epidermis, pancreatic progenitor cells, angioblasts or endothelial progenitor cells (EPCs), and blast cells. Biomarkers and methods for the identification, isolation, and culture of progenitor cells are known to those of skill in the art. The disclosure is not limited in this respect.

The term “population of cells,” as used herein, may refer to an individual cell or to a plurality of cells. In some embodiments, a population of cells comprises at least 10, at least 10², at least 10³, at least 10⁴, least 10⁵, at least 10⁶, at least 10⁷, least 10⁸, at least 10⁹, at least 10¹⁰, or more than 10¹⁰ cells. A population of cells may be homogeneous (also referred to as pure), or heterogeneous. Pure cell populations are preferred, e.g., cell populations consisting of 100% of the respective stem or progenitor cells. However, in some embodiments, a population of cells that comprises at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or at least 99% of the respective stem or progenitor cells can also be used.

In some embodiments, engineered hydrogels are provided herein that comprise stem or progenitor cells capable of differentiating into a desired cell type, for example a cell type that supports tissue regeneration. The terms “differentiation” and “differentiate” as used herein, refer to a cellular developmental process by which a cell becomes increasingly specialized, e.g., during development of an organism or in vitro, e.g., in response to an exogenous stimulus, such as a growth or differentiation factor. Stem cells, for example, may undergo differentiation to form more specialized progenitor cells which are more restricted in their developmental potential, and which, in turn, may differentiate into specialized cells, e.g., endothelial cells, skin cells, neural cells, or fibroblasts, which exhibit only a very narrow developmental potential, or are terminally differentiated in that they cannot further differentiate into any other cell type.

In some embodiments, engineered hydrogels or hydrogel-forming compositions are provided herein that comprise stem or progenitor cells capable of differentiating into a desired cell type in response to a growth factor, e.g., in a controlled-release form. The terms “differentiation in response to a growth factor” or “differentiate in response to a growth factor,” as used herein, refer to differentiation that is caused by exposure of the respective cell to a growth factor. A cell that differentiates in response to a growth factor, accordingly, is a cell that is responsive to the growth factor. For example, in some embodiments, an endothelial progenitor cell differentiates into an endothelial cell in response to VEGF and/or PDGF, as described in more detail elsewhere herein. In some embodiments, a neuronal stem cell or a neuronal progenitor cell differentiates in response to noggin, BMP, FGF, EGF, SHH, and/or BDNF. For example, in some embodiments, a neuronal stem cell differentiates into a neuronal progenitor cell in response to noggin and EGF. In some embodiments, a neuronal progenitor cell differentiates into a dopaminergic neuron in response to FGF8 and SHH. In some embodiments, a neuronal progenitor cell differentiates into a motor neuron in response to SHH (sonic hedgehog homolog) and RA (retinoic acid). In some embodiments, a neuronal stem cell differentiates into a glial progenitor cell in response to BMP. In some embodiments, a liver stem or progenitor cell differentiates into a hepatocyte in response to BMP and FGF. In some embodiments, a definitive endodermal cell differentiates into a pancreatic progenitor cell in response to activin A, Wnt3a and/or an inhibitor of SHH signaling (e.g., a small molecule inhibitor or an siRNA). Additional cell types that differentiate into a desired cell type in response to a growth factor or a combination of growth factors will be apparent to those of skill in the art, and the disclosure is not limited in this respect.

Accordingly, in some embodiments, engineered hydrogels or hydrogel-forming compositions are provided herein that comprise endothelial progenitor cells and VEGF as well as PDGF, e.g., in a controlled-release form. In some embodiments, engineered hydrogels or hydrogel-forming compositions are provided herein that comprise neuronal stem cells and noggin as well as EGF, e.g., in a controlled-release form. In some embodiments, engineered hydrogels or hydrogel-forming compositions are provided herein that comprise neuronal progenitor cells as well as FGF8 and SHH, e.g., in a controlled-release form. In some embodiments, engineered hydrogels or hydrogel-forming compositions are provided herein that comprise neuronal progenitor cells as well as SHH (sonic hedgehog homolog) and RA (retinoic acid), e.g., in a controlled-release form. In some embodiments, engineered hydrogels or hydrogel-forming compositions are provided herein that comprise neuronal stem cells and BMP, e.g., in a controlled-release form. In some embodiments, engineered hydrogels or hydrogel-forming compositions are provided herein that comprise liver stem or progenitor cells as well as BMP and FGF, e.g., in a controlled-release form. In some embodiments, engineered hydrogels or hydrogel-forming compositions are provided herein that comprise definitive endodermal cells as well as activin A, Wnt3a and/or an inhibitor of SHH signaling (e.g., a small molecule inhibitor or an siRNA), e.g., in a controlled-release form.

Accordingly, some embodiments provide engineered hydrogels or hydrogel-forming compositions that comprise a population of endothelial progenitor cells, e.g., of human ES-cell derived endothelial progenitor cells, and VEGF as well as PDGF, e.g., in a controlled-release form. Some embodiments provide engineered hydrogels or hydrogel-forming compositions that comprise a population of endothelial progenitor cells, e.g., of human ES-cell derived endothelial progenitor cells, and VEGF as well as PDGF, e.g., in a controlled-release form, and, additionally, a cell population of desired cells, e.g., cardiomyocytes, pancreatic cells, osteoblasts, neurons, neural progenitor cells, glial cells, fibroblasts, or keratinocytes. In some embodiments, such hydrogels and hydrogel-forming compositions are useful to graft desired cells into injured or damaged tissue in the form of a vascularized tissue patch. In some embodiments, such hydrogels and hydrogel-forming compositions further comprise one or more growth factors to which the additional cell population is responsive, e.g., BMP, VEGF, and EGF in the case of cardiomyocytes, BMP and 13FGF in the case of pancreatic cells, and FGF, VEGF, BMP2, and BMP4 in the case of osteoblasts.

Additional useful combinations of cells and growth factors that can be embedded into hydrogels or included in hydrogel-forming compositions will be apparent to those of skill in the art based on this disclosure. As the relations between stem or progenitor cells, growth factors, and desired cells are well known, those of skill in the art will be able to identify additional combinations of stem or progenitor cells and growth factors to induce differentiation yielding a desired cell or cell type. The disclosure is not limited in this respect.

The term “desired cell type,” as used herein, refers to a cell type that is of therapeutic benefit for a subject, for example, in that it regenerates a damaged or diseased tissue, or supports tissue regeneration, e.g., by providing mechanical or nutritional support or in clearing cellular debris from affected tissue (e.g., after cell death caused by ischemic injury). In some embodiments, a desired cell type may include, without limitation, exocrine secretory epithelial cells, hormone secreting cells, epithelial cells, e.g., epithelial cells lining closed internal body cavities, such as endothelial cells, keratinizing epithelial cells, stratified barrier epithelial cells, sensory transducer cells, neurons, glial cells, myelinating cells, hepatocytes (liver cells), adipocytes, lung epithelial cells, kidney cells, pancreatic cells (e.g., insulin-producing cells), intestinal brush border cells, fibroblasts, pericytes, odontoblasts, chondrocytes, osteoblasts, osteoclasts, muscle cells, cardiomyocytes, erythrocytes, megakaryocytes, monocytes, dendritic cells, microglial cells, leukocytes, T cells, B cells, melanocytes, germ cells, or interstitial cells. Additional cell types that confer a therapeutic benefit to a tissue or a subject experiencing tissue dysfunction will be apparent to the skilled artisan, and the present disclosure is not limited in this respect.

In some embodiments, engineered hydrogels are provided herein that comprise, embedded in the hydrogel scaffold, a population of stem or progenitor cells that is capable to differentiate into a desired cell type in response to a growth factor. The term “growth factor,” as used herein, refers to a substance capable of stimulating cellular growth, proliferation, and/or differentiation in cells that are responsive to the growth factor, for example, in cells that express a receptor that binds the growth factor. For example, an angiogenic growth factor may stimulate pro-angiogenic effects, e.g., growth and/or proliferation of cells that mediate angiogenesis, or differentiation of stem or progenitor cells, e.g., endothelial progenitor cells, into a cell type mediating angiogenesis, e.g., endothelial cells. Typically, a growth factor induces growth, proliferation, and/or differentiation through cellular signaling, e.g., through binding to a receptor on the surface of or within a responsive cell, which, in turn, effects downstream signaling causing cellular growth, proliferation, and/or differentiation. Most growth factors do not affect all cells or cell types of a subject, but induce growth, proliferation, and/or differentiation in a specific subset of cell types, or in only a single cell type that is responsive to the growth factor. A cell responsive to a specific growth factor, accordingly, is a cell capable of translating the presence of the growth factor into cellular growth, proliferation, and/or differentiation, e.g., a cell expressing a growth factor receptor able to bind the growth factor and effect growth factor-mediated downstream signaling.

Growth factors, growth factor receptors, the spectrum of cells responsive to a specific growth factor, and the effect of growth factors on the respective responsive cells, e.g., the effect on signaling pathways, gene expression patterns, and cellular responses such as growth, proliferation, and/or differentiation, are well known to those of skill in the art. Exemplary growth factors that are suitable for inclusion into engineered hydrogels, hydrogel-forming composition, and for use in the related methods described herein, include, without limitation, angiogenic growth factors (e.g., ERAP1, TYMP, EREG, FGF1, FGF2, FGF6, FIGF, IL18, JAG1, PDGF, PGF, TNNT1, VEGF, VEGFA, and VEGFC); apoptosis regulators (e.g., CLC, GDNF, IL10, IL1A, IL1B, IL2, NRG2, NTF3, SPP1, TDGF1, TGFB1, and VEGFA); cell differentiation factors (e.g., ERAP1, BMP1, BMP2, BMP3, BMP4, BMP5, BMP6, BMP7, BMP8B, CSF1, CSPG5, TYMP, EREG, FGF1, FGF2, FGF22, FGF23, FGF6, FGF9, FIGF, IL10, IL11, IL12B, IL2, IL4, INHA, INHBA, INHBB, JAG1, JAG2, LTBP4, MDK, NRG1, OSGIN1 (OKL38), PGF, SLCO1A2, SPP1, TDGF1, TNNT1, and VEGFC); developmental controllers (e.g., BMP10, NRG1, NRG2, NRG3, and TDGF1 (embryonic development); BDNF, CSPG5, CXCL1, FGF11, FGF13, FGF14, FGF17, FGF19, FGF2, FGF5, GDF11, GDNF, GP1, IL3, INHA, INHBA, JAG1, MDK, NDP, NRG1, NRTN, NTF3, PDGFC, PSPN, PTN, and VEGFA (nervous system development); FGF2, MSTN, HBEGF, IGF1, and TNNT1 (muscle development); GDF10, GDF11, IGF1, IGF2, INHA, and INHBA (skeletal development); BMP2, BMP3, BMP4, BMP5, BMP6, BMP7, BMP8B (cartilage development)); and others (e.g., AMH, CECR1, CSF2, CSF3, DKK1, FGF7, LEFTY1, LEFTY2, LIF, LTBP4, NGF, NODAL, TGFB1, THPO).

The structures and functions of these and other growth factors, as well as their spectrum of responsive cells, and their effect on their respective responsive cells, are well known to those of skill in the art and can be assessed, for example, in public databases such as the GenBank database (see, e.g., Benson D, et al. (2008). GenBank. Nucleic Acids Research 36 (Database): D25-D30. doi:10.1093/nar/gkm929. PMID 18073190, the entire contents of which are incorporated herein by reference), or the National Center for Biotechnology Information database (NCBI). A non-limiting list of exemplary growth factors that are suitable for inclusion into engineered hydrogels, hydrogel-forming composition, and for use in the related methods provided herein, together with references to their respective GenBank database entry accession numbers is provided in Table 1. Additional useful growth factors will be apparent to those of skill in the art. The disclosure is not limited in this respect.

TABLE 1
exemplary growth factors useful in the context of some embodiments of this disclosure.
The entire contents of each GenBank database entry listed, including, but not limited to, the
sequence of the growth factor-encoding nucleic acid molecule(s) and of the encoded growth
factor(s) described therein are incorporated herein by reference.

| Symbol | GenBank | Description | Gene Name |
|---|---|---|---|
| AMH | NM_000479 | Anti-Mullerian hormone | MIF, MIS |
| ERAP1 | NM_016442 | Endoplasmic reticulum aminopeptidase 1 | A-LAP, ALAP, APPILS, ARTS-1, |
| | | | ARTS1, ERAAP, ERAAP1, |
| | | | KIAA0525, PILS-AP, PILSAP |
| BDNF | NM_001709 | Brain-derived neurotrophic factor | MGC34632 |
| BMP1 | NM_006129 | Bone morphogenetic protein 1 | FLJ44432, PCOLC, PCP, PCP2, TLD |
| BMP10 | NM_014482 | Bone morphogenetic protein 10 | MGC126783 |
| BMP2 | NM_001200 | Bone morphogenetic protein 2 | BMP2A |
| BMP3 | NM_001201 | Bone morphogenetic protein 3 | BMP-3A |
| BMP4 | NM_130851 | Bone morphogenetic protein 4 | BMP2B, BMP2B1, MCOPS6, OFC11, |
| | | | ZYME |
| BMP5 | NM_021073 | Bone morphogenetic protein 5 | MGC34244 |
| BMP6 | NM_001718 | Bone morphogenetic protein 6 | VGR, VGR1 |
| BMP7 | NM_001719 | Bone morphogenetic protein 7 | OP-1 |
| BMP8B | NM_001720 | Bone morphogenetic protein 8b | BMP8, MGC131757, OP2 |
| CECR1 | NM_177405 | Cat eye syndrome chromosome region, | ADA2, ADGF, IDGFL |
| | | candidate 1 | |
| CLC | NM_001828 | Charcot-Leyden crystal protein | GAL10, Gal-10, LGALS10, |
| | | | LGALS10A, LPPL_HUMAN, |
| | | | MGC149659 |
| CSF1 | NM_000757 | Colony stimulating factor 1 (macrophage) | MCSF, MGC31930 |
| CSF2 | NM_000758 | Colony stimulating factor 2 (granulocyte- | GMCSF, MGC31935, MGC138897 |
| | | macrophage) | |
| CSF3 | NM_000759 | Colony stimulating factor 3 (granulocyte) | C17orf33, CSF3OS, GCSF, |
| | | | MGC45931 |
| CSPG5 | NM_006574 | Chondroitin sulfate proteoglycan 5 | MGC44034, NGC |
| | | (neuroglycan C) | |
| CXCL1 | NM_001511 | Chemokine (C—X—C motif) ligand 1 (melanoma | FSP, GRO1, GROa, MGSA, MGSA-a, |
| | | growth stimulating activity, alpha) | NAP-3, SCYB1 |
| DKK1 | NM_012242 | Dickkopf homolog 1 (Xenopus laevis) | DKK-1, SK |
| TYMP | NM_001953 | Thymidine phosphorylase | ECGF, ECGF1, MEDPS1, MNGIE, |
| | | | MTDPS1, PDECGF, TP, hPD-ECGF |
| EREG | NM_001432 | Epiregulin | ER |
| FGF1 | NM_000800 | Fibroblast growth factor 1 (acidic) | AFGF, ECGF, ECGF-beta, ECGFA, |
| | | | ECGFB, FGF-alpha, FGFA, GLIO703, |
| | | | HBGF1 |
| FGF11 | NM_004112 | Fibroblast growth factor 11 | FHF3, FLJ16061, MGC102953, |
| | | | MGC45269 |
| FGF13 | NM_004114 | Fibroblast growth factor 13 | FGF-13, FGF2, FHF-2, FHF2 |
| FGF14 | NM_004115 | Fibroblast growth factor 14 | FGF-14, FHF-4, FHF4, MGC119129, |
| | | | SCA27 |
| FGF17 | NM_003867 | Fibroblast growth factor 17 | FGF-13 |
| FGF19 | NM_005117 | Fibroblast growth factor 19 | — |
| FGF2 | NM_002006 | Fibroblast growth factor 2 (basic) | BFGF, FGFB, HBGF-2 |
| FGF22 | NM_020637 | Fibroblast growth factor 22 | — |
| FGF23 | NM_020638 | Fibroblast growth factor 23 | ADHR, HPDR2, HYPF, PHPTC |
| FGF5 | NM_004464 | Fibroblast growth factor 5 | HBGF-5, Smag-82 |
| FGF6 | NM_020996 | Fibroblast growth factor 6 | HBGF-6, HST2 |
| FGF7 | NM_002009 | Fibroblast growth factor 7 | HBGF-7, KGF |
| FGF9 | NM_002010 | Fibroblast growth factor 9 (glia-activating | GAF, HBFG-9, MGC119914, |
| | | factor) | MGC119915, SYNS3 |
| FIGF | NM_004469 | C-fos induced growth factor (vascular | VEGF-D, VEGFD |
| | | endothelial growth factor D) | |
| GDF10 | NM_004962 | Growth differentiation factor 10 | BMP-3b, BMP3B |
| GDF11 | NM_005811 | Growth differentiation factor 11 | BMP-11, BMP11 |
| MSTN | NM_005259 | Myostatin | GDF8 |
| GDNF | NM_000514 | Glial cell derived neurotrophic factor | ATF1, ATF2, HFB1-GDNF, HSCR3 |
| GPI | NM_000175 | Glucose-6-phosphate isomerase | AMF, DKFZp686C13233, GNPI, NLK, |
| | | | PGI, PHI, SA-36, SA36 |
| HBEGF | NM_001945 | Heparin-binding EGF-like growth factor | DTR, DTS, DTSF, HEGFL |
| IGF1 | NM_000618 | Insulin-like growth factor 1 (somatomedin C) | IGF-I, IGF1A, IGFI |
| IGF2 | NM_000612 | Insulin-like growth factor 2 (somatomedin A) | C11orf43, FLJ22066, FLJ44734, IGF- |
| | | | II, PP9974 |
| IL10 | NM_000572 | Interleukin 10 | CSIF, IL-10, IL10A, MGC126450, |
| | | | MGC126451, TGIF |
| IL11 | NM_000641 | Interleukin 11 | AGIF, IL-11 |
| IL12B | NM_002187 | Interleukin 12B (natural killer cell stimulatory | CLMF, CLMF2, IL-12B, NKSF, |
| | | factor 2, cytotoxic lymphocyte maturation factor | NKSF2 |
| | | 2, p40) | |
| IL18 | NM_001562 | Interleukin 18 (interferon-gamma-inducing | IGIF, IL-18, IL-1g, IL1F4, MGC12320 |
| | | factor) | |
| IL1A | NM_000575 | Interleukin 1, alpha | IL-1A, IL1, IL1-ALPHA, IL1F1 |
| IL1B | NM_000576 | Interleukin 1, beta | IL-1, IL1-BETA, IL1F2 |
| IL2 | NM_000586 | Interleukin 2 | IL-2, TCGF, lymphokine |
| IL3 | NM_000588 | Interleukin 3 (colony-stimulating factor, | IL-3, MCGF, MGC79398, MGC79399, |
| | | multiple) | MULTI-CSF |
| IL4 | NM_000589 | Interleukin 4 | BCGF-1, BCGF1, BSF-1, BSF1, IL-4, |
| | | | MGC79402 |
| INHA | NM_002191 | Inhibin, alpha | — |
| INHBA | NM_002192 | Inhibin, beta A | EDF, FRP |
| INHBB | NM_002193 | Inhibin, beta B | MGC157939 |
| JAG1 | NM_000214 | Jagged 1 | AGS, AHD, AWS, CD339, HJ1, |
| | | | JAGL1, MGC104644 |
| JAG2 | NM_002226 | Jagged 2 | HJ2, SER2 |
| LEFTY1 | NM_020997 | Left-right determination factor 1 | LEFTB, LEFTYB |
| LEFTY2 | NM_003240 | Left-right determination factor 2 | EBAF, LEFTA, LEFTYA, MGC46222, |
| | | | TGFB4 |
| LIF | NM_002309 | Leukemia inhibitory factor (cholinergic | CDF, DIA, HILDA |
| | | differentiation factor) | |
| LTBP4 | NM_003573 | Latent transforming growth factor beta binding | FLJ46318, FLJ90018, LTBP-4, |
| | | protein 4 | LTBP4L, LTBP4S |
| MDK | NM_002391 | Midkine (neurite growth-promoting factor 2) | FLJ27379, MK, NEGF2 |
| NDP | NM_000266 | Norrie disease (pseudoglioma) | EVR2, FEVR, ND |
| NGF | NM_002506 | Nerve growth factor (beta polypeptide) | Beta-NGF, HSAN5, MGC161426, |
| | | | MGC161428, NGFB |
| NODAL | NM_018055 | Nodal homolog (mouse) | MGC138230 |
| NRG1 | NM_013957 | Neuregulin 1 | ARIA, GGF, GGF2, HGL, HRG, |
| | | | HRG1, HRGA, MST131, NDF, SMDF |
| NRG2 | NM_013982 | Neuregulin 2 | DON1, HRG2, NTAK |
| NRG3 | NM_001010848 | Neuregulin 3 | HRG3, pro-NRG3 |
| NRTN | NM_004558 | Neurturin | NTN |
| NTF3 | NM_002527 | Neurotrophin 3 | HDNF, MGC129711, NGF-2, NGF2, |
| | | | NT3 |
| OSGIN1 | NM_182981 | Oxidative stress induced growth inhibitor 1 | BDGI, OKL38 |
| PDGFC | NM_016205 | Platelet derived growth factor C | FALLOTEIN, SCDGF |
| PGF | NM_002632 | Placental growth factor | D12S1900, PGFL, PLGF, PIGF-2, |
| | | | SHGC-10760 |
| PSPN | NM_004158 | Persephin | PSP |
| PTN | NM_002825 | Pleiotrophin | HARP, HBGF8, HBNF, NEGF1 |
| SLCO1A2 | NM_021094 | Solute carrier organic anion transporter family, | OATP, OATP-A, OATP1A2, SLC21A3 |
| | | member 1A2 | |
| SPP1 | NM_000582 | Secreted phosphoprotein 1 | BNSP, BSPI, ETA-1, MGC110940, |
| | | | OPN |
| TDGF1 | NM_003212 | Teratocarcinoma-derived growth factor 1 | CR, CRGF, CRIPTO |
| TGFB1 | NM_000660 | Transforming growth factor, beta 1 | CED, DPD1, LAP, TGFB, TGFbeta |
| THPO | NM_000460 | Thrombopoietin | MGC163194, MGDF, MKCSF, ML, |
| | | | MPLLG, TPO |
| TNNT1 | NM_003283 | Troponin T type 1 (skeletal, slow) | ANM, FLJ98147, MGC104241, STNT, |
| | | | TNT, TNTS |
| VEGFA | NM_003376 | Vascular endothelial growth factor A | MGC70609, MVCD1, VEGF, VPF |
| VEGFC | NM_005429 | Vascular endothelial growth factor C | Flt4-L, VRP |

In some embodiments, engineered hydrogels are provided that comprise the growth factors VEGF and/or PDGF.

The terms “platelet-derived growth factor” and “PDGF,” as used herein, refer to a family of growth factors encoded by the four genes PDGFA, PDGFB, PDGFC. The encoded proteins can form disulfide-linked homodimers referred to as PDGF AA, PDGF BB, PDGF CC, and PDGF DD, and the heterodimer PDGF AB (see, e.g., Li, X. and U. Eriksson (2003) Cytokine &Growth Factor Rev. 14:91, the entire contents of which are incorporated herein by reference). PDGF is a potent angiogenic factor, and PDGF growth factors are expressed in multiple embryonic and adult cell types and tissues. It stimulates vascular smooth muscle cell proliferation and may play an important role in cardiovascular development and function (see, e.g., Gilbertson, D. et al. (2001) J. Biol. Chem. 276:27406, the entire contents of which are incorporated herein by reference). Without wishing to be bound by any particular theory, it is believed that PDGF family growth factors are primarily involved in angiogenesis (the growth of blood vessels from pre-existing vasculature).

The terms “vascular endothelial growth factor” and “VEGF,” as used herein, refer to a sub-family of growth factors comprising a cysteine-knot motif (see, e.g., 4. Robinson, C. J. and S. E. Stringer (2001) J. Cell. Sci. 114:853; Leung, D. W. et al. (1989) Science 246:1306; Keck, P. J. et al. (1989) Science 246:1309; and Byrne, A. M. et al. (2005) J. Cell. Mol. Med. 9:777; the entire contents of each of which are incorporated herein by reference). The VEGF family includes VEGFA, VEGFB, VEGFC, VEGFD, and PIGF. In some embodiments, the term VEGF refers to VEGFA, also known as vascular permeability factor (VPF). VEGF is a potent mediator of both angiogenesis and vasculogenesis in the fetus and adult. Humans express alternately spliced isoforms of 121, 145, 165, 183, 189, and 206 amino acids (aa) in length, and VEGF₁₆₅ appears to be the most abundant and potent isoform, followed by VEGF121 and VEGF189. Without wishing to be bound by any particular theory, growth factors of the VEGF sub-family are believed to be primarily involved in vasculogenesis (the de novo formation of the embryonic circulatory system) and are also believed to play a supportive role in angiogenesis (the growth of blood vessels from pre-existing vasculature).

In some embodiments, the engineered hydrogels provided comprise the growth factors VEGF and PDGF, and a population of cells responsive to these growth factors, for example, a population of endothelial progenitor cells.

In some embodiments, engineered hydrogels are provided that comprise a growth factor in a controlled-release form or a controlled-release formulation. The terms “controlled-release form” and “controlled-release formulation,” as used herein, refer to a formulation of an agent from which the agent is released in a predictable manner, or following predictable kinetics. Typically, a controlled-release form of an agent to be released, e.g., of a growth factor, comprises the agent associated with a carrier, e.g., bound to a solid support or encapsulated in a carrier. For example, a controlled-release formulation of a growth factor may, in some embodiments, comprise the growth factor associated with a carrier, and the growth factor is released from the carrier by dissociating from it. In some embodiments, the association may be via non-covalent interactions, e.g., via ionic bond or van der Waals forces. In some embodiments, the growth factor may be encapsulated in the carrier, and be released from the carrier as the carrier dissolves or disintegrates. In some embodiments, the carrier dissolves over time, thus releasing the agent, e.g., the growth factor associated with it. In other embodiments, the agent is released from the carrier upon a stimulus, e.g., a shift in pH or temperature, or exposure to an agent cleaving a bond between the agent and the carrier, e.g., an enzyme, a reactive moiety, or light.

In some embodiments, a controlled-release form provides a supply of the agent to be released, e.g., a growth factor, from which the agent is released over time. Controlled release may be rapid or slow, may be continuous over time (e.g., following zero-order kinetics), may fluctuate over time, or may be in one or multiple waves. Some methods and compositions for the formulation of controlled-release forms, for example, controlled-release forms of growth factors, are described in more detail elsewhere herein.

The skilled artisan will be able to ascertain numerous controlled-release formulations that are suitable for use in the context of embedding a growth factor into a hydrogel described herein, as well as methods and compositions for the preparation of such controlled-release forms for use in the context of some embodiments of this disclosure, e.g., in the context of controlled release of growth factors. Such controlled-release formulations, methods, and compositions include, for example, those disclosed in Donald Wise, Handbook of Pharmaceutical Controlled Release Technology, CRC Press; 1st edition (Aug. 15, 2000), ISBN-10: 0824703693; Herbert Lieberman, Pharmaceutical Dosage Forms: Disperse Systems, Volume 3, Informa Healthcare; 2nd edition (Jan. 15, 1998) ISBN-10: 0824798422; Juergen Siepmann, Ronald Siegel, and Michael Rathbone (eds.), Fundamentals and Applications of Controlled Release Drug Delivery (Advances in Delivery Science and Technology), Springer; 2012 edition (Dec. 14, 2011), ISBN-10: 1461408806; and Chapter 6, pages 177-212, of Ajay Banga, Therapeutic Peptides and Proteins: Formulation, Processing, and Delivery Systems, Second Edition, CRC Press; 2 edition (Sep. 14, 2005), ISBN-10: 0849316308; the entire contents of each of which are incorporated herein by reference. It will be understood that other controlled-release forms may also be suitable for use in some embodiments of this disclosure and that the disclosure is not limited in this respect.

In some embodiments, engineered hydrogels or hydrogel-forming compositions are provided that comprise a growth factor in a controlled-release form. In some embodiments, the controlled-release form is a liposome encapsulating the growth factor. The term “liposome,” as used herein, refers to a vesicle comprising a core surrounded by a lipid layer, typically by a lipid bilayer. In some embodiments, the core is liquid, for example, comprising an aqueous solution comprising the respective growth factor. In some embodiments, the core is solid, e.g., comprising a solid matrix, particle, granule, or powder comprising the respective growth factor. In some embodiments, liposomes comprise phospholipids in the lipid layer. In some embodiments, liposomes are multilamellar vesicles (MLVs), small unilamellar vesicles (SUVs), or large unilamellar vesicles (LUVs). In some embodiments, liposomes are used as controlled-release forms of growth factors. Typically, such liposomes comprise a liquid core, e.g., an aqueous solution containing the growth factor. Depending on the hydrophilicity of the growth factor, the growth factor may be comprised in the liquid core, at the intersection of the liquid core and the lipid layer, or in the lipid layer of a liposome.

Liposome-encapsulated controlled-release forms are particularly suitable for the controlled release of hydrophilic growth factors in aqueous solution. Methods and reagents for encapsulating a growth factor, e.g., VEGF or PDGF in aqueous solution, into a liposome are well known to those of skill in the art. In some embodiments, the liposomes comprise a DMPC or DMPG lipid bilayer. As described in more detail elsewhere herein, these lipids have a relatively low melting Temperature™, and thus create liposomes with a high bilayer fluidity, which, in turn, results in a quick release, or a high rate of release, of the encapsulated growth factor. DMPC or DMPG liposomes are, accordingly particularly suitable for the delivery of growth factors directing the initial stages of differentiation of a responsive stem or progenitor cell, e.g., such as VEGF in the case of endothelial progenitor cell differentiation. In some embodiments, the liposomes comprise a DSPC or DSPG lipid bilayer. As described in more detail elsewhere herein, these lipids have a relatively high Tm, and thus create liposomes with a low bilayer fluidity, which, in turn, results in a slow release, or a low rate of release, of the encapsulated growth factor. DSPC or DSPG liposomes are particularly suitable for the delivery of growth factors directing later stages of differentiation of a responsive stem or progenitor cell, or are required or beneficial for an extended period of time, such as PDGF in the case of endothelial progenitor cell differentiation.

The release kinetics of a liposome-entrapped or liposome-encapsulated growth factor depend on, among other factors, the fluidity of the lipid layer, which, in turn, depends on, among other factors, the melting temperature (Tm) of the lipid(s) in the lipid layer of the liposome. For example, liposomes with relatively fluid lipid layers can be produced using 1,2-distearoyl-sn-glycero-3-phosphocholine (DSPC) and/or 1,2-distearoyl-sn-glycero-3-phosphatidylglycerol (DSPG), with a Tm of about 56° C., and liposomes with relatively rigid lipid layers can be produced using 1,2-dimyristoyl-sn-glycero-3-phosphocholine (DMPC), and/or 1,2 dimyristoyl-sn-glycero-3-phosphatidylglycerol (DMPG), with a Tm of about 23° C. Typically, DSPC/DSPG liposomes will release an encapsulated growth factor at a higher rate of release than the DMPC/DMPG liposomes based on their higher lipid layer fluidity.

Suitable methods for producing liposomes encapsulating growth factors for controlled release include, but are not limited to, thin lipid film hydration methods, as described in more detail elsewhere herein. Additional methods and materials suitable for the generation of liposomes encapsulating of proteins, e.g., growth factors, that are useful in the context of this disclosure are known to those of skill in the art, and such materials and methods include, without limitation, those described in Vladimir Torchilin, Liposomes: a practical approach, Oxford University Press, USA; 2 edition (Aug. 7, 2003), ISBN-10: 0199636540; Gregory Gregoriadis, Liposome Technology, Volume I: Liposome Preparation and Related Techniques, Third Edition, Informa Healthcare (Sep. 12, 2006), ISBN-10: 084938821X; and Gregory Gregoriadis, Liposome Technology, Volume II: Entrapment of Drugs and Other Materials into Liposomes, Third Edition, Informa Healthcare; (Sep. 12, 2006), ISBN-10: 0849388287; the entire contents of each of which are incorporated herein by reference. Additional suitable materials and methods will be apparent to the skilled artisan based on this disclosure. The disclosure is not limited in this respect.

Some of the most important characteristics of a controlled-release form of a growth factor as provided herein are its release kinetics. The term “release kinetics,” as used herein, refers to the kinetics of release of an agent, e.g., a growth factor, from a controlled-release form. In some embodiments, the release kinetics are zero-order kinetics, in which the agent is released at the same rate of release from a controlled-release form over time. In some embodiments, the release kinetics feature a decreasing or increasing rate of release over time, or a burst or wave of release, e.g., in response to a stimulus, such as a change in pH or temperature, or to exposure to a bond-cleaving enzyme, ionizing radiation, or light. The term “rate of release,” as used herein in the context of controlled-release formulations, refers to the amount of an agent (e.g., the number of molecules or the mass of agent) that is released from a controlled-release form within a given time frame. The rate of release may, e.g., be expressed as number of molecules released per minute, hour, or day, e.g., mol/min, mol/h, or mol/day. Alternatively, the rate of release may be expressed as the mass of the agent released per minute, hour, or day, e.g., ng/min, ng/h, μg/day, and so forth, or as the percentage of encapsulated agent over time, e.g., %/min, %/hr, %/120 hr, and so forth. In some embodiments, a growth factor is provided in a controlled-release form that exhibits a rate of release of about 1 fg/h-10 μg/h (e.g., about 10 pg/h-10 ng/h, about 100 pg/h-10 ng/h, about 100 fg/h-10 pg/h, or about 1 ng/h-1 μg/h) per encapsulated mg of growth factor. In some embodiments, a growth factor is provided in a controlled-release form that exhibits a rate of release of about 1 fmol/h-10 μmol/h (e.g., about 10 pmol/h-10 nmol/h, about 100 pmol/h-10 ng/h, about 100 fmol/h-10 pmol/h, or about 1 nmol/h-1 μmol/h) per encapsulated mmol of growth factor. In some embodiments, a growth factor is provided in a controlled-release form that exhibits a rate of release of about 0.0005%/h-1%/h (e.g., about 0.001%/h-0.01%/h, about 0.001%/h-0.1%/h, about 0.0005%/h-0.001% g/h, or about 0.01%/h-1%/h) of the total amount of encapsulated growth factor.

In some embodiments, engineered hydrogels and hydrogel-forming compositions are provided that comprise a growth factor, e.g., a growth factor described in Table 1, and a population of cells that is responsive to the growth factor, e.g., a population of cells that are able to differentiate into a desired cell type for tissue regeneration, such as cells that can form or regenerate blood vessels, neurons, or muscle cells when exposed to the growth factor. In some preferred embodiments, the growth factor, for example, a growth factor described in Table 1, is provided in the hydrogel in a controlled-release form, for example, encapsulated into a liposome. Some of the hydrogels provided herein, accordingly, comprise a population of stem or progenitor cells and a growth factor that the cells are responsive to, embedded in the hydrogel scaffold. The co-embedding of a growth factor and growth factor responsive cells has the advantage that the resulting close proximity of the source of the growth factor and the responsive cells allows for highly efficient direction of cell differentiation at the site of administration. One advantage is that the proximity of growth factor source and responsive cells allows for the use of dosages of growth factors that are much lower than dosages that would be needed in the case of systemic administration. The use of controlled-release forms of growth factors further avoids the need for repeated administration of growth factors that are required to be present over extended periods of time in order to direct appropriate cellular differentiation.

The use of controlled-release forms of growth factors embedded in some of the engineered hydrogels provided herein, or comprised in some of the hydrogel-forming compositions provided herein, further allow for the generation of complex growth factor signaling patterns, e.g., sequential exposure of the encapsulated cells to a plurality of growth factors, or exposure to different concentrations of different growth factors, or shifting concentrations of different growth factors over time. The choice of different controlled-release forms with suitable release kinetics for the delivery of different growth factors, in combination with the close proximity of the source of the growth factors and the responsive cells, allows one of skill in the art to mimic virtually any pattern of growth factor exposure that may be required in order to direct differentiation of the encapsulated stem or progenitor cells into a desired cell type.

Accordingly, some embodiments provide engineered hydrogels or hydrogel-forming compositions that comprise a plurality of growth factors. In some embodiments, at least two growth factors comprised in the hydrogel or the hydrogel-forming composition are in different controlled-release forms. In some embodiments, the different controlled-release forms exhibit different release kinetics, for example, in that the different controlled-release forms exhibit different rates of release. In some embodiments, a growth factor is released from the respective controlled-release form at a high rate of release, e.g., at a rate of more than 0.1%, more than 0.15%, more than 0.2%, more than 0.25%, more than 0.3%, more than 0.4%, more than 0.5%, more than 0.6%, more than 0.7%, more than 0.75%, more than 0.8%, more than 0.9%, or more than 1% of encapsulated growth factor released per hour. In some embodiments a growth factor is released from the respective controlled-release form at a low rate of release, e.g., at a rate of less than 0.1%, less than 0.09%, less than 0.08%, less than 0.075%, less than 0.05%, less than 0.025%, less than 0.01%, less than 0.005%, less than 0.001%, or less than 0.0005% of encapsulated growth factor released per hour. In some embodiments, two growth factors are embedded in a hydrogel provided herein, e.g., VEGF and PDGF, or any two growth factors described in Table 1, and one is released quickly, e.g., from a controlled-release form with a high rate of release, and the other is released slowly, e.g., from a controlled-release form with a low rate of release. For example, in some embodiments, an engineered hydrogel or hydrogel-forming composition is provided that comprises VEGF and PDGF-responsive cells, e.g., endothelial progenitor cells, and VEGF in a controlled-release form that releases the VEGF quickly, as well as PDGF in a controlled-release form that releases the PDGF slowly.

Some aspects of this disclosure provide hydrogel-forming compositions. In some embodiments, a hydrogel-forming composition comprises components that can form a hydrogel, e.g., a hydrogel as described herein. Such components may include, for example, polymers that can form a hydrogel scaffold. In some embodiments, hydrogel-forming compositions provided herein also include cells and/or growth factors that can be embedded in the hydrogel to be formed. A hydrogel-forming composition as provided herein typically does not comprise a hydrogel scaffold, but functionalized polymers that, when brought into contact with each other under suitable conditions, can react to form a hydrogel scaffold. The functionalized polymers are typically provided separately or in the absence of a component required for the formation of a covalent bond between the polymers, e.g., in case where the polymers are inert under certain conditions, e.g., in the absence of a catalyst or a source of energy, a hydrogel-forming composition may be provided under such conditions of inertia.

For example, in some embodiments, a hydrogel-forming composition is provided herein that comprises a growth factor, e.g., VEGF and/or PDGF, or a growth factor described in Table 1, in a controlled-release form; a polymer, e.g., a polysaccharide such as DEX, CMC, or HA, comprising a first reactive moiety; and a polymer, e.g., a polysaccharide such as DEX, CMC, or HA, comprising a second reactive moiety that forms a covalent bond with the first reactive moiety under physiological conditions, thus forming a hydrogel. In some embodiments, the composition also comprises a population of stem or progenitor cells that differentiates into a desired cell type in response to the growth factor, e.g., a population of endothelial progenitor cells that differentiate into endothelial cells in the presence of VEGF and PDGF. Similar to the hydrogels provided herein, a hydrogel-forming composition may comprise a plurality of growth factors. In some embodiments, at least two growth factors comprised in the hydrogel-forming composition are in different controlled-release forms, for example, in different controlled-release forms that exhibit different release kinetics. In some embodiments, the polymers are provided in aqueous solution, for example, in separate aqueous solutions in the case of polymers carrying reactive moieties that form covalent bonds under physiological conditions. In some embodiments, at least one of the aqueous solutions is suitable for the culture or compatible with at least short-term survival of cells and/or biological stability of a growth factor in a controlled-release form. In some such embodiments, a population of cells and/or a growth factor in a controlled-release form is comprised in such an aqueous polymer solution. In some embodiments, the separate aqueous solutions comprising the reactive polymers and, optionally, the population of cells and/or the growth factor, are combined before or upon injection or implantation of the composition into a subject. In some such embodiments, the combining of the solutions results in covalent crosslinking of the polymers and the formation of a hydrogel. In embodiments where a growth factor and/or a population of cells is comprised in one of the polymer solutions, the resulting hydrogel scaffold will encapsulate these components, forming a hydrogel comprising a growth factor.

In some embodiments, the hydrogel-forming composition comprises a multi-compartment container holding the reactive polymer solutions in separate compartments. For example, in some embodiments, the container is a multi-compartment syringe comprising one reactive polymer in one compartment and another reactive polymer in another compartment. In some embodiments, the container, e.g., the syringe, comprises a nozzle for mixing the reactive polymers. In embodiments where the reactive polymers are mixed before administration, e.g., in a syringe with a mixing nozzle as described above, the mixture is typically administered to a tissue or a site of tissue injury before the bond-forming reaction is complete, in order to allow for in situ formation of the respective hydrogel scaffold. In some embodiments, suitable hydrogel-forming reactions may take seconds to minutes or even about an hour to complete under physiological conditions. Some suitable reactions and chemistries for pre-administration mixing of separate gel-forming components comprised in a hydrogel-forming composition are described herein. Additional suitable methods will be apparent to those of skill in the art. The disclosure is not limited in this respect.

Some embodiments provide hydrogels and hydrogel-forming compositions that can be used in vitro, for example, to differentiate stem or progenitor cells into differentiated cells and cell structures. Such in vitro differentiated structures can be used to study cellular differentiation and assembly processes, and the resulting engineered tissue constructs can be used as therapeutics. In some embodiments, such in vitro produced, hydrogel-embedded cellular structures or tissues are used as tissue patches, e.g., as a vascular patch to provide rapid relief of an ischemic condition, a skin patch to restore lost skin tissue, or as a neuronal patch to restore neuronal activity to a site of injury of the central nervous system.

Some aspects of this disclosure provide engineered hydrogels or hydrogel-forming compositions that can be used to treat acute or chronic lack of tissue perfusion, including acute ischemia, hypoxia, or anoxia, e.g., in the context of stroke, myocardial ischemia, peripheral arterial disease (PAD), claudication, hind limb ischemia, and any disease characterized by blood vessel occlusion or loss of function.

The terms “treat,” “treating,” and “treatment,” as used herein refer to a clinical intervention intended to ameliorate a clinical symptom of a disease or disorder. This may include, in some embodiments, ameliorating a clinically manifest symptom, e.g., a symptom of loss of function of a tissue, e.g., of tissue vascularization, tissue homeostasis, or other tissue function, and may also include, in some embodiments, the prevention or inhibition of progression of a disease or disorder. In some embodiments, treatment includes administering a therapeutic composition to a subject in need of such treatment. In some embodiments, treatment includes tissue regeneration, e.g., restoration, full or in part, of a function that was lost or impaired in a tissue, such as tissue perfusion, vascularization, or specialized tissue function (e.g., brain function, muscle function, vasculature function).

Some hydrogels provided herein that are useful for the treatment of a disease, disorder, or tissue dysfunction in a subject comprise a population of stem or progenitor cells capable of differentiating into a cell type forming blood vessels, e.g., endothelial progenitor cells that can differentiate into blood vessel-forming endothelial cells in the presence of appropriate molecular cues. In some embodiments, the hydrogel also comprises the appropriate molecular cues in the form of a growth factor in a controlled-release form. For example, in some hydrogels provided for use in this context, VEGF is comprised in a controlled-release form exhibiting a high rate of release, and PDGF in a controlled-release form exhibiting a low rate of release. After administration of such a hydrogel to a site of injury to a blood vessel causing tissue ischemia, the epithelial progenitor cells will be exposed to an initial burst of VEGF, which is believed to be beneficial for vasculogenesis, or the differentiation of epithelial progenitor cells into epithelial cells and the subsequent formation of new blood vessels, and a sustained release of PDGF, which stabilizes the newly formed blood vessels, and supports angiogenesis.

In the context of treatment, hydrogels and/or hydrogel-forming compositions provided herein are administered in an effective amount. In general, an effective amount is any amount that can cause a beneficial change in a desired tissue, e.g., a regeneration of lost tissue, or a restoration, full or in part, of a tissue function. In some embodiments, an effective amount is an amount sufficient to cause a beneficial change in a particular disease or disorder, e.g., an alleviation of a symptom caused by acute ischemia, e.g., in the context of stroke, or of a symptom caused by a chronic disease, such as PAD. In general, an effective amount is that amount of a pharmaceutical preparation that alone, or together with further doses, produces a desired response. This may involve slowing the progression of a disease or disorder temporarily, halting the progression of a disease or disorder permanently, delaying or preventing the onset of a disease or disorder, or reversing one or more symptoms of a disease or disorder. Hydrogels and hydrogel-forming compositions are typically administered locally at the site of tissue injury or into or adjacent to dysfunctional tissue. The dosage of the hydrogels and hydrogel-forming compositions provided herein will depend on the nature of the tissue to be treated, and also on the nature and extent of the injury or dysfunction at hand. Single-dose applications are typically preferred, in particular in embodiments, where application is performed during surgery. However, in some embodiments, repeated application of a hydrogel or hydrogel-forming composition may be required. In such embodiments, minimally-invasive or non-invasive administration routes are preferred. In general, a single application of a hydrogel or hydrogel-forming composition will be in the range of 0.1 mg-10 g in total weight, but extensive tissue damage, e.g., large-scale burns, may necessitate larger quantities, e.g., in the range of 10 g-1 kg. Where cells are included in the hydrogel or hydrogel-forming composition, the number of cells per administration may be between 10-10¹⁰ cells in some embodiments, and preferably about 10³, about 10⁴, about 10⁵, about 10⁶, or about 10⁷ cells per dose. Larger amounts of cells may be administered, if necessary. In some embodiments, the effective amount is not determined by the total amount of hydrogel or hydrogel-forming composition, but by the amount of stem or progenitor cells and/or growth factors comprised in the hydrogel or the hydrogel-forming composition.

### Methods

Some aspects of this disclosure provide therapeutic methods comprising administering a hydrogel or a hydrogel-forming composition as described herein to a subject in need thereof. In some embodiments, the subject is a subject in need of tissue regeneration. In some embodiments, a subject in need of administration of a hydrogel or a hydrogel-forming composition as described herein or a subject in need of tissue regeneration is a subject suffering from or diagnosed with a tissue dysfunction, for example, a loss of function of a tissue or a loss of tissue, e.g., of brain tissue, spinal cord tissue, vasculature, muscle tissue, liver tissue, kidney tissue, or pancreatic tissue. Such a tissue dysfunction or tissue loss may be associated with acute trauma, e.g., acute severance or partial severance of the spinal cord, acute ischemia as a result of arterial occlusion or disruption or myocardial infarction, or with other types of tissue injury, e.g., abrasion, cuts, toxin exposure, burn, or ionizing radiation. Tissue loss or dysfunction may also be associated with a chronic disease or disorder, e.g., PAD, claudication, an autoimmune disease, such as type I diabetes, or a neurodegenerative disease. In some embodiments, the hydrogel or hydrogel-forming composition comprises a growth factor, e.g., a growth factor described in Table 1, in a controlled-release form, and a population of cells capable of regenerating the dysfunctional, injured, or lost tissue in the presence of the growth factor.

The term “subject,” as used herein, refers to an individual organism, for example, a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig.

In some embodiments, the subject is a subject in need of revascularization of a tissue, e.g., after stroke, myocardial infarction, or arterial occlusion or severance. In some such embodiments, a therapeutic method is provided that comprises administering to the subject a hydrogel or a hydrogel-forming composition that comprises endothelial progenitor cells, VEGF in a controlled-release form exhibiting a high rate of release, and PDGF in a controlled-release form exhibiting a low rate of release. In some embodiments, the hydrogel or the composition is administered into or in direct proximity to the site of injury, e.g., to cover, or wrap around the occluded or severed artery. In some embodiments, the method includes monitoring the subject after administration for clinical signs of ischemia, hypoxia, anoxia, or necrosis in the affected tissue.

Some aspects of this disclosure provide methods for generating a hydrogel, e.g., in a clinical or non-clinical context. In some embodiments, the method comprises providing a growth factor in a controlled-release form; a polymer comprising a first reactive moiety; and a polymer comprising a second reactive moiety that forms a covalent bond with the first reactive moiety under physiological conditions; and contacting the polymers with each other in the presence of the growth factor under physiological conditions. The result will be, in some embodiments, the formation of a hydrogel encapsulating the growth factor in the controlled-release form. In some embodiments, the method further comprises providing a population of stem or progenitor cells that differentiates into a desired cell type in response to the growth factor. For example, in some embodiments, the growth factor is VEGF and PDGF, in a quick-release form and a slow-release form, respectively, and endothelial progenitor cells. In some such embodiments the contacting of the polymers is in the presence of the growth factor and of the cells, with the result being the formation of a hydrogel encapsulating the growth factor and the cells.

In some embodiments, the polymers are provided separately in aqueous solutions for injection, and in some such embodiments, the cells and/or the growth factor are suspended in one of the aqueous polymer solutions, either together or separately. In some embodiments, the reactive moieties comprised in the polymers are click chemistry moieties, and the contacting is performed under conditions suitable for the respective click chemistry reaction to take place. In some embodiments, the first reactive moiety is an aldehyde moiety, the second reactive moiety is an adipic anhydride moiety, and the covalent bond being formed upon the contacting of the polymers with each other is a hydrazone bond. In some such embodiments, the polymers are contacted with each other under physiological conditions. In some embodiments, the method comprises administering a hydrogel-forming composition as provided herein to a subject, wherein the polymers are contacted with each other immediately prior to, upon, or immediately subsequent to administration. In some embodiments comprising administering a hydrogel-forming composition to a subject, the result of the administering is the formation of a hydrogel at the site of administration. For example, in some embodiments, a hydrogel-forming composition comprising reactive polymers, e.g., reactive polysaccharides as described herein, VEGF in a quick-release form, PDGF in a slow-release form, and endothelial progenitor cells, is administered to an occluded artery of a subject at the site of artery occlusion. The result of this administration, in some embodiments, is the formation of a hydrogel comprising VEGF and PDGF in their respective release forms and of endothelial progenitor cells at the site of artery occlusion. In some embodiments, the endothelial progenitor cells differentiate into endothelial cells and form new blood vessels that bypass the arterial occlusion, thus improving or preventing at least one symptom associated with the arterial occlusion, such as acute ischemia, hypoxia, anoxia, hemorrhage, cell death or necrosis.

### Kits

Some aspects of this disclosure provide kits comprising components or reagents useful for the generation of hydrogels as described herein or for the administration of hydrogels or hydrogel-forming compositions as described herein to a subject. In some embodiments, such kits provide the components needed by a health practitioner to practice the therapeutic methods provided herein. For example, in some embodiments, the kit comprises a polymer comprising a first reactive moiety; and a polymer comprising a second reactive moiety, wherein the second reactive moiety forms a covalent bond with the first reactive moiety under physiological conditions, thus forming a hydrogel. In some embodiments, the kit also comprises a growth factor in a controlled-release form, for example, a growth factor described in Table 1 in a controlled-release form, e.g., VEGF in a quick-release form and PDGF in a slow-release form. In some embodiments, the kit further comprises a population of cells that differentiate into a desired cell type in response to the growth factor, e.g., a population of endothelial progenitor cells. In some embodiments, the kit further comprises an applicator for administering the components of the kit to a subject, or for generating the hydrogel in vitro. In some embodiments, the applicator comprises separate compartments for holding an aqueous solution comprising one of the reactive, hydrogel-forming polymers each. In some embodiments, the applicator also comprises a mixer, for example, a mixing nozzle, for mixing and/or administering the aqueous solutions. In some embodiments, the kit comprises a plurality of growth factors in different controlled-release forms, for example, VEGF and PDGF in a release form having a high rate of release and a release form having a low rate of release, respectively. In some embodiments, the controlled-release form is a liposome-encapsulated form. In some embodiments, the liposomes in which the growth factors are encapsulated are selected from the group consisting of DMPC liposomes (high rate of release) and DSPC liposomes (low rate of release). In some embodiments, the kit comprises a population of endothelial progenitor cells.

The function and advantage of these and other embodiments of the present disclosure will be more fully understood from the Examples below. The following Examples are intended to illustrate the benefits of the present disclosure and to describe particular embodiments, but are not intended to exemplify the full scope of the disclosure and, accordingly, do not limit the scope of the disclosure.

### EXAMPLE

### In Situ Forming Hydrogels for the Treatment of Ischemic Tissue

Hydrogels comprising growth factors and stem or progenitor cells were developed and evaluated for their therapeutic application for vascular generation. Hydrogels comprising growth factors that stimulate embryonic vascular development were generated and their utility in tissue engineering and therapy is demonstrated herein. Injectable hydrogels were developed that comprised in situ cross-linked polysaccharides (e.g., HA, DEX, and/or CMC) and growth factors (e.g., VEGF and PDGF) entrapped in liposomes. Culturing endothelial progenitor cells (EPC) derived from human ES cells on growth factor-eluting hydrogels directed their in vitro differentiation into a vascular network. In vivo formation of this vasculature in an ischemic hind limb mouse model protected the ischemic limb from necrosis and restored the functionality of the vasculature to nearly normal blood perfusion.

### Introduction

Peripheral arterial disease (PAD) is associated with high morbidity and significant impairment of quality of life in over 25% of world population [1-3]. This disease is caused by critical, typically atherosclerotic, narrowing or blockage of the arteries that supply blood to the internal organs and extremities. Reduced vascular perfusion of the affected tissues results in tissue ischemia. Ischemic manifestation ranges from painful cramping of the limbs to limb ulceration and amputation-requiring gangrene, depending on the severity of the vascular occlusion. PAD patients with underlying risk factors (e.g. diabetes mellitus, hyperlipidemia, hypertension) have a 20%-30% risk of limb amputation [1,3]. Current treatment options fail to reduce this risk [2, 3]. While surgical bypass of the vascular occlusion is frequently impossible because of the complex vascular anatomy, administration of angiogenic growth factors (e.g. vascular endothelial growth factor (VEGF), fibroblasts growth factor (FGF) and hepatocyte growth factor) led to disappointing results in clinical trials [2-4]. New approaches are needed to develop more efficacious treatment modalities. Controlled tissue neovascularization could present an alternative for ischemia therapy. Neovascularisation is a process involving vessel formation (vasculogenesis) and their subsequent sprouting (angiogenesis).

Recently, endothelial progenitor cell (EPC)-based approaches have shown promise for guiding tissue neovascularisation [5]. Adult EPCs have been isolated from the bone marrow, spleen, cord blood and circulating cells in peripheral blood of adult humans [6-10]. These adult endothelial progenitors have been shown to home to sites of new blood vessels and contribute to functional vasculature, leading to potential therapeutic applications such as cell transplantation for repair of ischemic tissue and tissue engineering of vascular grafts [10-14]. However, recent in vivo evidence points to low homing and engraftment efficiencies [2, 15]. In addition, the low amounts of EPC present in peripheral blood and bone marrow might pose therapeutic limitations specifically in patients suffering myocardial infarction [16].

Human ESCs are advantageous as a source of endothelial cells or endothelial progenitor cells when compared with other sources of endothelial cells, due to their high proliferation capability, pluripotency, and low immunogenity [17]. Recent findings indicated that a subpopulation of vascular progenitor cells, isolated from hESCs, has the ability to differentiate to endothelial-like and smooth muscle like cells, depending on the choice of supplemented growth factor (VEGF and platelet derived growth factor (PDGF), respectively) [18]. These two growth factors are intimately involved in the process of vascularization. However, it is not only the presence of these two factors that influences angiogenesis, but also their temporal presentation. VEGF is responsible for the initiation of angiogenesis and involves endothelial cell activation and proliferation, while PDGF is required after VEGF activation in order to allow for blood vessel maturation through recruitment of smooth muscle cells [19].

Some aspects of this disclosure are based on the recognition that spatiotemporally controlled presentation of vasculogenic and angiogenic growth factors can mimic the process of vascular development during embryogenesis and assist the formation of a functional 3D vascular network. To this end, growth factor-eluting polysaccharide-based hydrogels were developed that allow for the spatiotemporal controlled presentation of growth factors. VEGF and PDGF were incorporated into polysaccharide based hydrogels through entrapment in liposomes. Liposomal entrapment was chosen because the liposome physiochemical properties such as charge and lipid composition can be utilized to construct tailor-made carriers for temporal secretion of these growth factors [19-21]. To overcome the current hurdles associated with injecting EPCs into systemic circulation and the associated loss of control over their fate and destination, hESC-derived EPCs were delivered to the site of injury in a mouse model of hindlimb ischemia using injectable hydrogels. An EPC subpopulation of CD34-positive cells was chosen for hydrogel entrapment since these cells have been shown to differentiate into either endothelial cells or smooth muscle cells depending on the choice of growth factor supplementation [18]. By combining cell and growth factor delivery, two goals were achieved: 1) EPCs were directed to differentiate into blood vessels and 2) neovascularization was imparted on host cells while EPCs formed new blood vessels. The EPC-laden injectable hydrogels developed here were shown to significantly improve ischemia outcome and reduce limb necrosis and autoamputation.

### Results

Composite injectable hydrogels comprised of in situ cross-linked polysaccharides and liposomes containing growth factors (VEGF and PDGF) were developed. These composite systems were characterized in regards to gelation time and swelling, microstructure and cytotoxicity. Liposome physiochemical properties were varied to achieve the desired release kinetics, in this case a high rate of release of VEGF and a low rate of release of PDGF. Once the desired microenvironment properties were established, the potential of growth factor-eluting hydrogels in inducing vascularization of hESCs both in vitro and in vivo in a hind limb ischemia model was evaluated.

Hydrogel Preparation and Characterization.

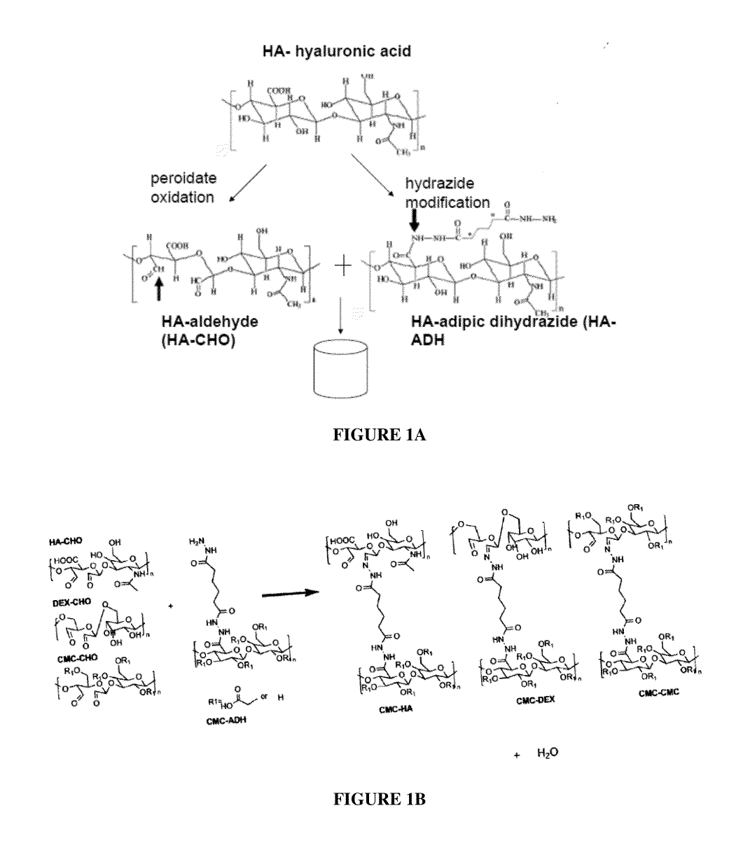

To form injectable in situ crosslinking hydrogels, polysaccharides were chemically modified, exploiting the reactivity of their carboxy and hydroxy groups. Carboxymethylcellulose (CMC), hyaluronic acid (HA) and dextran (DEX) were modified with aldehyde functionality (-CHO) by periodate oxidation or by hydrazide modification with adipic anhydride functionality (-ADH). See FIG. 1A for an illustration of HA functionalization. ¹H NMR spectra of CMC-ADH [22] demonstrated that 50% of the N-acetyl-D-glucosamine residues were modified, as calculated from the ratio of the area of the peak for the N-acetyl-D-glucosamine residue of CMC (singlet peak at 2.0 ppm) to that for the methylene protons of the adipic dihydrazide at 1.62 ppm. Analysis of aldehyde groups formed by the oxidation of dextran with hydroxylamine yielded a 33% degree of oxidation.

When CHO and ADH modified polysaccharide derivatives were mixed, they reacted to form a cross-linked hydrogel through formation of hydrazone bonds, and water as the only by-product (FIG. 1B). The various CHO-polysaccharides were combined with CMC-ADH by placing them in separate syringes in a double-barreled syringe holder (Table 2). Cross-linked polysaccharides are denoted throughout by hyphenated abbreviations, e.g. HA-CHO/CMC-ADH.

TABLE 2
Modified polysaccharide concentrations

| | Polymer weight % in 1 ml |
|---|---|
| Polymer | PBS solution |
| HA-ADH | 1, 2.5, 6 |
| DEX-ADH | 6 |
| CMC-ADH | 2.5 |
| HA-CHO | 2 |
| DEX-CHO | 6 |

The effects of incorporating liposomes and cells into the hydrogels on the gelation time and swelling of hydrogels was examined. The average gelation time of DEX-CHO/CMC-ADH gels was ˜30 sec at 25° C., and was accelerated by the incorporation of liposomes; no difference was observed by addition of cells. Increasing the temperature from 25 to 37° C. accelerated gelation time (p<0.001 between all groups tested and between the groups at different temperatures, Table 3). In term of swelling properties, the DEX-CHO/CMCADH hydrogel also had the lowest swelling ratio: 53±4% compared to 231±26% for HA-CHO/HA-ADH and 128±31% for dextran-CHO/HA-ADH in PBS at 37° C. Incorporation of liposomes or cells didn't increase the swelling of DEX-CHO/CMCADH hydrogels. For HA-CHO/HA-ADH hydrogels, swelling increased following cell incorporation by 20%, while incorporation of liposomes alone didn't change swelling. For DEX-CHO/HA-ADH hydrogels, swelling increased by less than 10% by addition of cells, with no observed change with the addition of liposomes alone.

TABLE 3
Gelation times of different hydrogels (sec)

| Temp | | No | | | Liposomes |
|---|---|---|---|---|---|
| ° C. | Composition | Additive | Liposomes | Cells | and cells |
| 25 | HA-CHO/HA-ADH | 5 ± 0.6 | 4.6 ± 0.7 | 4.8 ± 0.8 | 4.6 ± 0.5 |
| | DEX-CHO/HA-ADH | 7 ± 0.4 | 6.5 ± 0.6 | 6.9 ± 0.5 | 6.7 ± 0.8 |
| | DEX-CHO/CMC-ADH | 32 ± 0.8 | 25.1 ± 1 | 23.8 ± 1.2 | 25.8 ± 0.6 |
| 37 | HA-CHO/HA-ADH | 4 ± 0.6 | 3.6 ± 0.7 | 3.8 ± 0.8 | 3.6 ± 0.5 |
| | DEX-CHO/HA-ADH | 5.6 ± 0.2 | 5.1 ± 0.4 | 5.3 ± 0.6 | 5.3 ± 0.8 |
| | DEX-CHO/CMC-ADH | 24 ± 0.3 | 17 ± 1.5 | 16.3 ± 0.7 | 16.3 ± 0.7 |

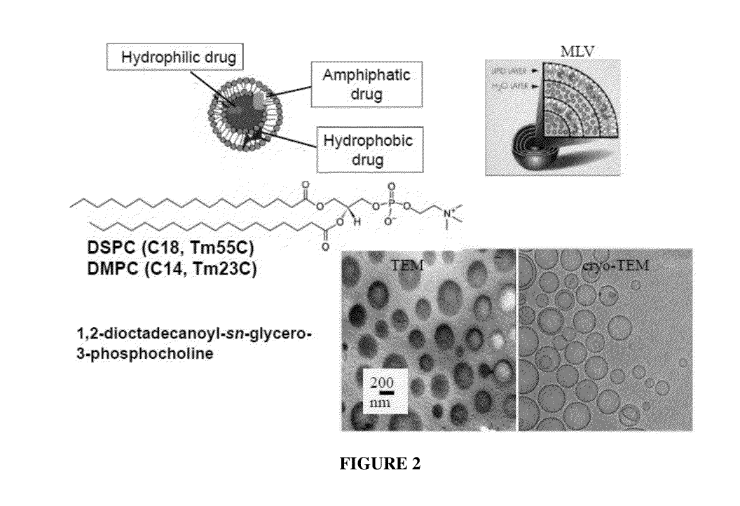

Release kinetics of growth factors from cross-linked hydrogels were investigated. VEGF and PDGF were readily encapsulated in liposomes forming 4±1.3 μm sized particles. Differences in growth factor hydrophilicity (VEGF is more hydrophilic than PDGF) resulted in lower encapsulation efficiency of VEGF compared to PDGF (46%±7% VEGF was encapsulated in DSPC, 43%±5% VEGF was encapsulated in DMPC, 61%±5% PDGF was encapsulated in DSPC, 56%±7%) PDGF was encapsulated in DMPC. FIG. 2 shows a schematic of liposome structure and size distribution of some liposome populations.

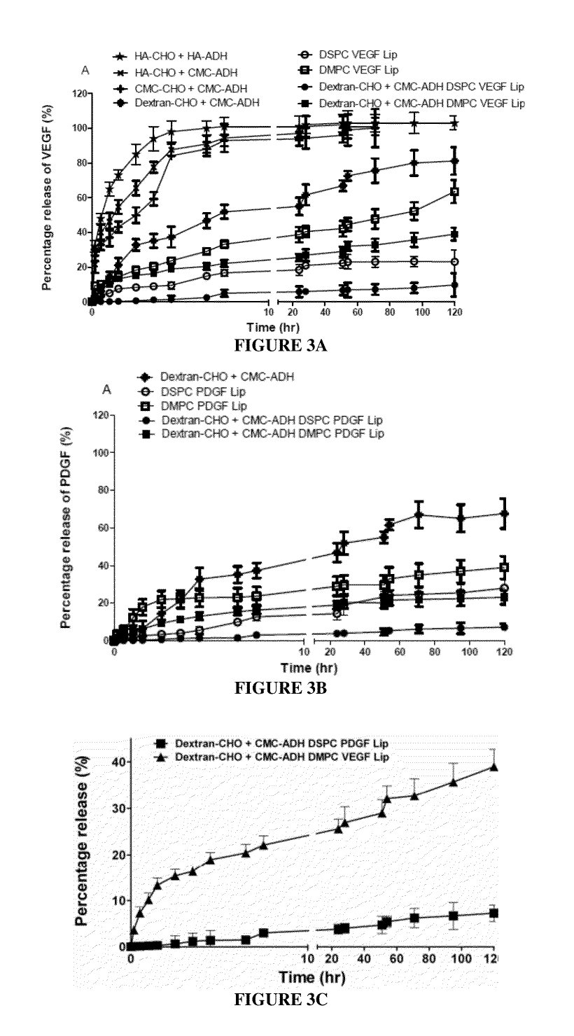

To identify a hydrogel composition capable of releasing VEGF and/or PDGF with selected target release rates (bolus and slow, respectively), in vitro release kinetics studies were performed in hESC media at 37° C. (FIG. 3). As a first attempt, VEGF was encapsulated with the different hydrogels (FIG. 3A). However, release cannot be controlled in this configuration as after 5 hours almost 100% of the growth factor was released from all examined hydrogels. Only upon encapsulation in liposomes (DMPC or DSPC) was a controllable release profile of VEGF achieved. Release of VEGF from DMPC liposomes displayed a slight burst effect, releasing 10% of the encapsulated VEGF within the first 30 min followed by a relatively constant release of 63% of the rest of encapsulated VEGF within 120 hr, corresponding to a release rate of 0.24% VEGF/hr. A different release profile was observed when VEGF was encapsulated in DSPC. From this high melting temperature liposome (Tm=55 C), no burst effect was observed, but rather a low, constant release rate of 0.15% VEGF/hr, leading to release of 29% of encapsulated VEGF within 120 hr. Thus, the release rate of VEGF from a fluid, low melting temperature DMPC liposome is faster than its release from a less fluid, high melting temperature DSPC liposome. Similar release profiles were measured for each liposome encapsulated within the hydrogels. Yet, embedding the liposomes within hydrogels further constrained the growth factor release rate. As shown in FIG. 3A, 40% of VEGF encapsulated in DMPC is released within 120 hr while only 10% of VEGF encapsulated in DSPC is released within 120 hr.

Similar trends were also observed in the release kinetics of PDGF from the same liposomes and hydrogels (FIG. 3B). That is, a burst effect releasing 20% of encapsulated PDGF from low melting temperature (Tm=37° C.) DMPC liposomes within the first hour followed by a constant release of 40% of the rest of encapsulated PDGF within 120 hr. PDGF exhibited a slower release rate than VEGF from the same liquid-like DMPC liposome due to its increased hydrophobicity compared to VEGF. No burst effect was observed for PDGF encapsulated within DSPC liposomes, but rather a steady release leading to 30% of encapsulated PDGF to be released within 120 hr. Incorporating these liposomes within hydrogels further constrained the release of PDGF. Thus, 22% of PDGF encapsulated in DMPC was released within 120 hr while only 10% of PDGF encapsulated in DSPC is released within 120 hr.

Hydrogels comprising a DEX-CHO/CMC-ADH scaffold and both VEGF in DMPC liposomes and PDGF in DSPC liposomes were generated. The release kinetics of the growth factors from these gels is shown in FIG. 3C.

In Vitro Vessel Formation.

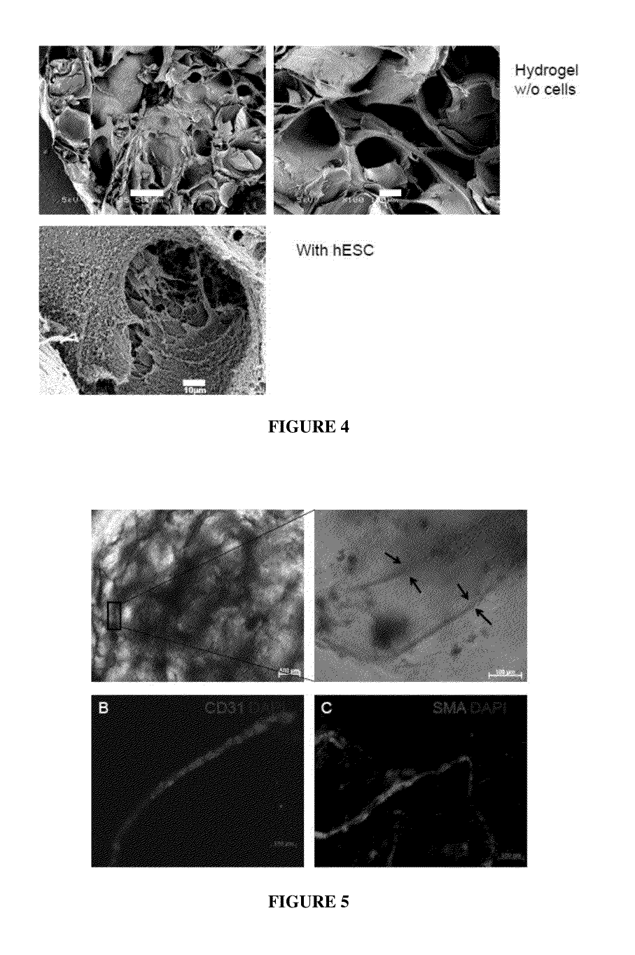

SEM images of lyophilized hydrogels indicate a porous network, distributed throughout the entire hydrogel disk (FIG. 4). This structure is typical for cross-linked hydrogels. [23] The porous network inside the hydrogels was necessary for survival of the seeded hESC. Human ESCs were able to form colonies which grew and survived within the hydrogels (FIG. 4). Viable cells, labeled with fluorescent green calcein (Live/Dead assay, Invitrogen) were detected through the entire depth of hydrogel disk following 5 days in culture. Of the various hydrogel compositions examined, DEX-CHO/CMC-ADH was the only combination to maintain integrity in media for prolonged time periods (longer than two weeks) when seeded with hESCs. All other hydrogel compositions tested, e.g., HA-CHO/HA-ADH and DEX-CHO/HA-ADH, decomposed within 2 and 5 days respectively, resulting in leakage of cells from hydrogels. Incorporation of hESCs in these hydrogels accelerated their decomposition as, without cells, degradation in media was observed to begin at day 5 and day 10, respectively. This observation could be explained by previous findings indicating that hESC secrete hyaluronidase enzymes which catalyze decomposition of hyaluronic-based hydrogels [24].

The effect of growth factors on hESC differentiation within the composite hydrogels was evaluated. To this end, CD34 positive cells were isolated, representing a vascular subset of endothelial progenitor cells, and seeded in hydrogels comprising liposome-encapsulated VEGF and PDGF. During two weeks in culture, the progenitor cells formed branched structures, similar to vascular networks (FIG. 5). Based on positive staining for both CD31 endothelial marker, and smooth muscle actin (SMA), a marker for smooth muscle cells, the branch-like network was determined to be composed mainly of endothelial and smooth muscle cells. In contrast, no network development was seen with bolus addition of the same growth factors.

In Vivo Functionality in Hind Limb Ischemia Model.

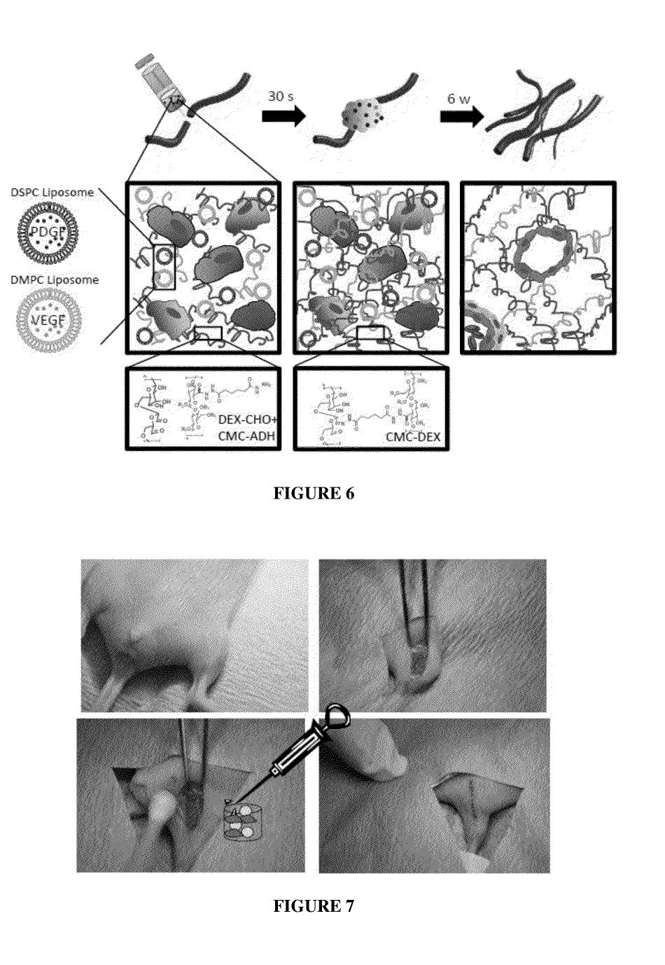

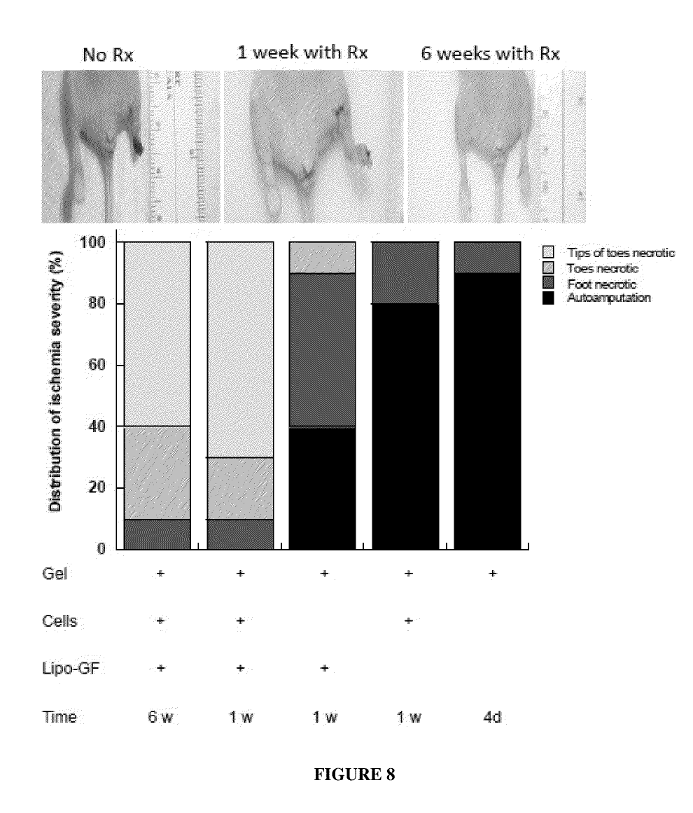

The functionality of the engineered branch like vascular network was examined in vivo using a mouse model of hind limb ischemia. Following generation of ischemia via femoral artery ligation, cell and growth factor-laden hydrogel was injected at the site of injury. A schematic of the procedure is shown in FIG. 6 and pre-surgery, peri-surgery- and post-surgery images are shown in FIG. 7. In the group of animals receiving growth factor-eluting, CD34+ cell-laden hydrogels, a hydrogel-forming composition comprising CHO and ADH-functionalized polysaccharides, liposome-encapsulated VEGF and PDGF, and CD34+ endothelial progenitor cells, was injected and cross-linked in situ through hydrazone bond formation at 37° C. Gel solidification occurred instantly. Five different treatments were compared for their ability to relieve ischemia. In all four control groups (n=8 for each), i.e., groups administered with CD34 positive cells, CD34 positive cell-laden hydrogels and growth factor-laden hydrogels, the mice developed severe limb necrosis within 2-3 days after ischemic injury (FIG. 8). In these groups necrosis progressed rapidly above ⅓ of the metatarsal bone and resulted in limb loss. In sharp contrast, ischemic mice injected with hESC-derived CD34 positive cells integrated within growth factor-eluting hydrogels developed only marginal necrosis, usually at the edges of digits, and the limb was salvaged (FIG. 8).

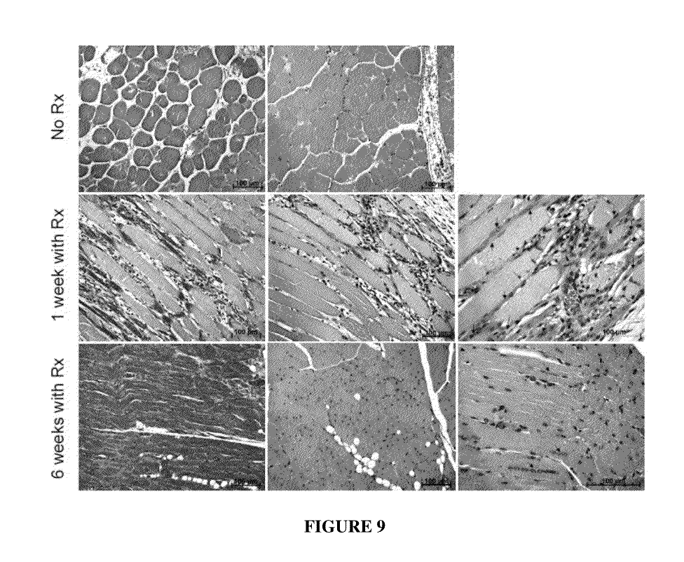

Histological examination of tissue harvested from the latter group (6 weeks after ischemic injury) revealed that the muscle bed was mostly composed of regenerated muscle fibers (FIG. 9). Regenerated muscle appeared as multi-nucleated muscle fibers with centered nucleus (as opposed to typical normal muscle fibers where the nucleus is usually at the fiber circumference). These regenerated areas were densely populated with small capillaries. Fibrosis (as measured by Trichrome staining) was minimal in this treated group and similar to normal muscle. In the control groups, the majority of the muscle bed was comprised of dead enucleated muscle fibers which stained blue/gray by Trichrome. Since the control group animals had to be euthanized within 1 week, histological examination of mice treated with CD34 positive cells and growth factor eluting hydrogels was performed 1 week after ischemic injury. Although the majority of the muscle bed was comprised of enucleated fibers, in between these dead fibers newly regenerated thin fibers appeared, distinctive by their multiple nuclei. These regenerated fibers, comprising about 20% of the entire muscle bed, appeared as red fibers as opposed to blue/gray dead fibers following Trichrome staining. Interestingly, 1 week after injury, inflammation (the appearance of monocytes) in the vicinity of the hydrogel was reduced in animals containing hydrogels with only cells or with cells and liposomes) compared to hydrogels containing only liposomes.

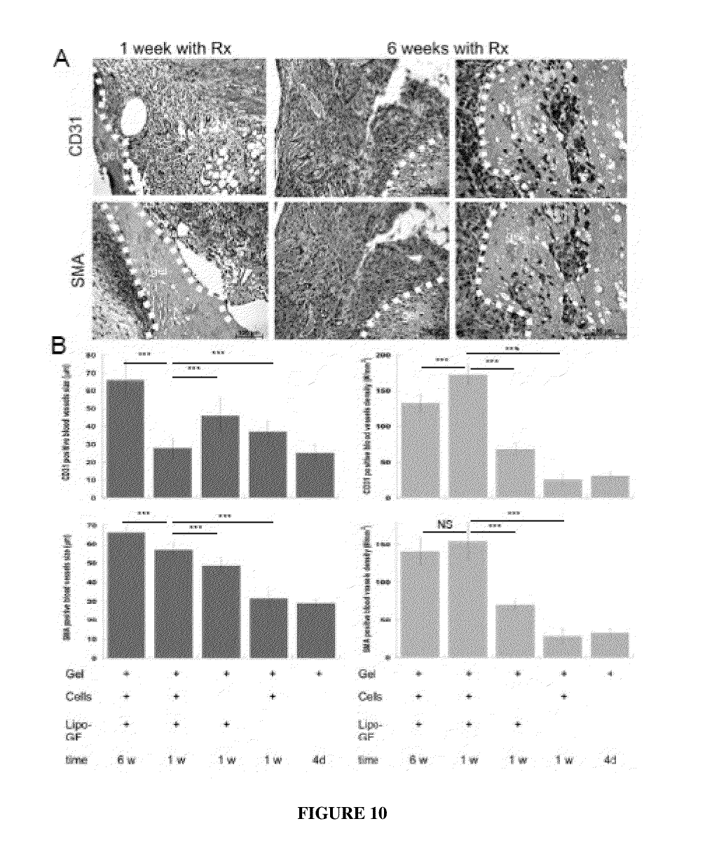

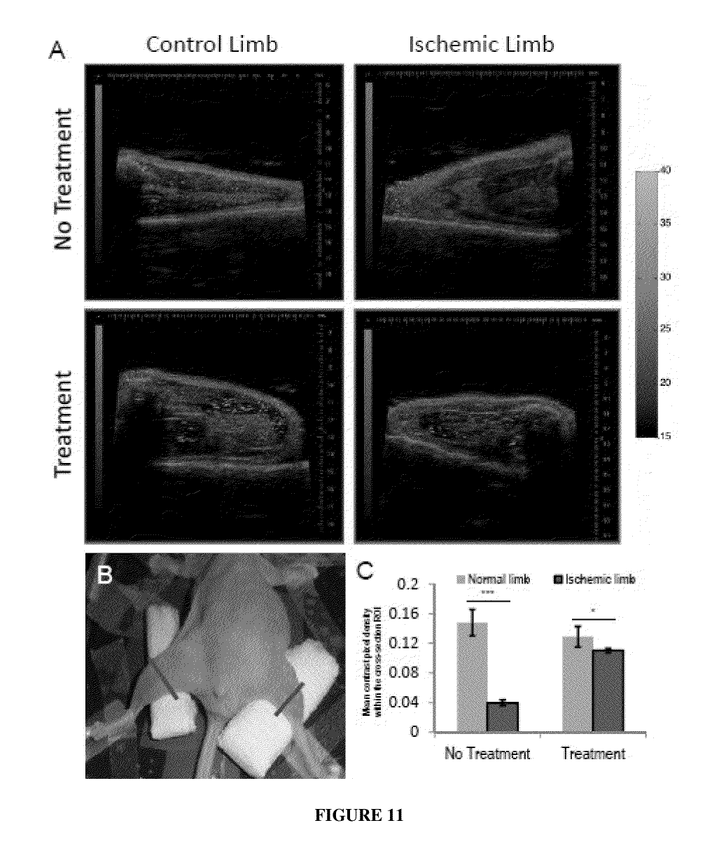

To examine neovascularization, staining against CD31 and SMA, indicative of endothelial cells and fibroblasts forming vessels, was conducted in 1 week treated mice and compared to the 6 week treated animals (FIG. 10). The 6 weeks-treated mice exhibited high density blood capillaries stained positively for both CD31 (131 vessels/mm²) and SMA 147 vessels/mm²) in the vicinity of and inside the hydrogels. Moreover, CD31 and SMA staining were colocalized, indicating the maturity of these blood vessels. All blood vessels appeared to have blood cells in them, indicative of their functionality. In addition, increased CD31 positive capillaries were observed in areas of regenerated muscle fibers. These phenomena of regenerated muscle fibers filled with capillaries already started in the 1 week treated mice. However, at this time point, no capillaries were observed in the hydrogels, but instead significant positive staining for SMA positive cells appeared in close proximity to hydrogels (FIG. 10). CD31 positive cells forming small capillaries were detected in the vicinity of the hydrogels but these had no blood cells inside. These were not colocalized with SMA staining. Yet still, blood vessels density was 2-3 higher than in mice treated with growth factor-eluting hydrogels (but without hESC-derived CD34+ cells) and 6-4 times higher than in mice treated with hESC derived CD34+ cell-laden hydrogels or any of the controls.

Blood perfusion of the ischemic limb was examined using ultrasound imaging in combination with intravenously administered microbubbles (FIG. 11). Microbubbles are strong reflectors of ultrasound energy and, thereby, provide powerful contrast enhancement on ultrasound images. Due to their relatively large size, microbubbles (2-4 μm) are purely intravascular flow tracers. Thus, visible contrast enhancement of a region of interest on an ultrasound scan, following intravascular microbubble administration, reflects regional perfusion. FIG. 11 shows pseudo-color ultrasound contrast scans of both the injured (ischemic) and control mouse hind limbs. As shown in the figure, while the ischemic limb of a control animal that did not receive treatment exhibited significantly diminished contrast enhancement, the ischemic hind limb treated with CD34+-cell-laden and growth factor-eluting hydrogel displayed contrast enhancement that was visually indiscernible from that exhibited by the healthy non-treated contralateral limb of the same animal. These data qualitatively confirm regeneration of the perfusion-supporting vascular bed in the ischemic limb upon treatment with hydrogels comprising both liposome-encapsulated VEGF and PDGF and hESC-derived CD34+ cells.

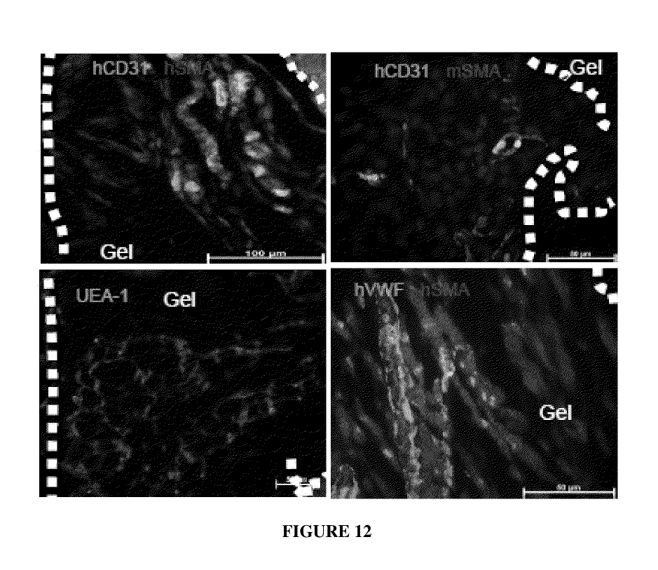

Expression of human endothelial markers was evaluated in hydrogels and their vicinity in the mice that received hydrogels comprising both hESC-derived CD34+ cells and liposome-encapsulated VEGF and PDGF after 6 weeks (FIG. 12). The hydrogels contained vascular structures that stained positively for human CD31, human αSMA, human Von Willebrand factor (VWF) and that bound Ulex Europaeus Agglutinin I (UEA-1), a marker for human endothelial cells. The positive staining for these markers indicates vessel maturation and that the cellular source of neovascularization within the gel was the hESC derived cell population embedded in the hydrogel, and not endogenous host cells migrating into the hydrogels. For vessels outside of the hydrogel, co-localization of human CD31⁺ and murine SMA staining (upper left image) indicated that cells embedded in the hydrogel were able to migrate into the host tissue, where they interacted with endogenous host cells to form new vasculature and to connect the vasculature formed in the hydrogel to the host's vasculature.

### Discussion

Injectable hydrogel-forming compositions were developed that are useful for the in situ generation of growth-factor-eluting, cell-laden hydrogels that can mimic the spatiotemporal variation pattern of biochemical signals for vascular differentiation, and thereby stimulate hESC-derived EPCs to form functional vascular networks. A functional vascular system is essential for the formation and maintenance of most tissues in the body, and the lack of vascularization results in ischemic tissues with limited intrinsic regeneration capacity. Engineering or regenerating a vascular network holds great promise in many therapeutic applications as it restores cell viability during growth of the tissue, induces structural organization, and can promote integration of artificial tissue constructs upon implantation and repair ischemic tissues.

The formation of the first capillaries takes place during early stages of embryogenesis through the process of vasculogenesis, the in situ assembly of capillaries from precursor endothelial cells [25]. In this process endothelial cells are generated from precursor cells. These then aggregate and establish cell-to-cell contacts, leading to formation of a nascent endothelial tube. A primary vascular network is then established from an array of such endothelial tubes [26]. Expansion of the network occurs via angiogenesis, referring to the formation of new capillaries from preexisting ones [27]. Vessel formation and subsequent stabilization requires multiple paracrine and autocrine signals. Among these signals, VEGF is a prominent one, secreted by cells surrounding the vessels and acting upon endothelial cells during their aggregation and tube formation [28]. Other factors, such as PDGF, are released by endothelial cells and act upon themselves and surrounding mesenchymal cells to stabilize the vascular network [29].

To mimic embryonic vascular development, a hydrogel was engineered that releases VEGF relatively fast (40%/120 hr) and that releases PDGF over a longer period of time. Based on the release kinetics, fast secretion of VEGF was achieved by encapsulation in DMPC liposomes and slow secretion of PDGF was achieved by encapsulation in DSPC liposomes. These liposomes were embedded into a DEX-CHO/CMC-ADH-based hydrogel scaffold. Among the hydrogel compositions examined, the DEX-CHO/CMC-ADH based hydrogel exhibited optimal properties, including preservation of integrity in media for periods longer than 5 days, and high cell viability. The engineered hydrogels provided herein allow for the tailoring of the kinetics at which encapsulated hESC-derived cells will be exposed to vasculogenesis and angiogenesis-directing growth factors, thus allowing to mimic the process of vascular development during embryogenesis both in vitro and in vivo. In vitro culturing of CD34 positive-hESC derived cells in these hydrogels resulted in the formation of a vessel-like network. The network was composed of endothelial and smooth muscle cells which are the building blocks of blood vessels. Importantly, the encapsulated CD34+ cells self-assembled into vascular structures in which endothelial cells were surrounded by smooth muscle cells. This resembles mature blood vessel structures where endothelial cells line the inner surface of blood vessels as an interface between the circulating blood and the adjacent smooth muscle layers.

The functionality of inducing the formation of such vascular networks was evaluated in a clinically relevant model of mouse hind limb ischemia. It was observed that in 7 out of 8 mice injected with hESC-derived CD34 positive cells integrated within growth factor eluting hydrogels, the ischemic limb was salvaged (ischemia was restricted to digits) and blood flow returned to normal values. In all control groups severe necrosis developed within 2-3 days post-surgery.

The in vivo potential of hESC-derived EPCs to treat ischemic tissues has been examined by several groups [30-32]. Although promising, these studies showed only minor improvements in ischemia treatment. Injection of a Von Willebrand factor (VWF) positive sub population of EPCs into hind limb ischemic mice resulted in only 36% limb salvation [30], while injection of an expanded VE-cadherin positive population of EPC improved blood perfusion by only 10% compared to control groups injected with PBS [31]. Further improvement in blood perfusion up to 30% was detected when these cells were co injected with an SMA positive subpopulation of EPC [32]. It should be noted that in these two aforementioned studies [31, 32] ischemia was induced by femoral vein ligation, leading to improvement in blood perfusion in control PBS injected mice. A more clinically relevant ischemia model employs femoral artery ligation (as used in this study) [30, 33].

One of the problems with injecting EPCs into systemic circulation (or even into the site of injury) is loss of control over their fate and destination. The hydrogels and methods developed here, on the other hand, use an injectable in situ cross-linked hydrogel to deliver EPCs into the site of injury and confine them there, as part of a combined approach to treat ischemia with both locally confined cells and growth factors.

Since it was not clear from the literature which cell type, e.g., which specific vascular identity or differentiation degree would provide the highest vasculogenic potential [17], a relatively immature CD34 positive EPC subset was chosen for the studies described herein. It was observed that differentiation of these immature cells into endothelial and smooth muscle cells and organization into a vascular network can be achieved by spatiotemporal control of VEGF and PDGF exposure.

Thus, the injectable hydrogels and hydrogel-forming compositions provided herein have a dual purpose: direct differentiation of CD34 positive EPCs into blood vessels as well as impart neovascularization on host cells by elution of growth factors from the hydrogels and interaction of the encapsulated progenitor cells with host cells, as shown in FIG. 12. Once vessels are formed they also in turn have a neovascularization effect on the surrounding host tissue. In summary, by combining hydrogel structures with controlled growth factor delivery and hESC-derive progenitor cells, a microenvironment was created that can induce differentiation into a vascularized substitute within a time frame and with a flow capacity sufficient to rescue acute ischemia. The in vivo formation of such a network induced neovascularization in hindlimb ischemic mice, prevented ischemia and salvaged a limb from necrosis and autoamputation.

### Materials and Methods

Liposome Preparation and Characterization.

Liposomes were prepared by modified thin lipid film hydration [34], using the following lipids: 1,2-distearoyl-sn-glycero-3-phosphocholine (DSPC), 1,2-distearoyl-snglycero-3-phosphatidylglycerol, (DSPG) and 1,2 dimyristoyl-sn-glycero-3phosphocholine (DMPC), all purchased from Genzyme (Cambridge, Mass.). These lipids were selected to produce relatively fluid (DMPC-DMPG) or solid (DSPC-DSPG) liposomes at 37° C. (phase transition temperatures, Tm; DSPC=56° C. and DMPC=23° C.). The liposomes' Tm will, thereby, influence the release kinetics on entrapped growth factors, resulting in a high rate of release from liposomes exhibiting high fluidity of the lipid layer(s) as opposed to a low rate of release from liposomes exhibiting less fluidity of their lipid layer(s). DSPC:DSPG:cholesterol or DMPC:DMPG:cholesterol (molar ratio 3:1:2) were dissolved in t-butanol (Riedel-de Hacn, Seelze, Germany). PDGF (Recombinant Human PDGF BB, CF, Cat #220-BB-050, R&D Systems, MN, USA) was added in DMPC before lyophilization. For the DSPC liposomes the lyophilized cake was hydrated with VEGF (Recombinant Human VEGF 165, Cat #293-VE-050, R&D Systems, MN, USA) in PBS buffer, at 55-60° C. and for DMPC liposomes the lyophilized cake was hydrated with PBS, at 37-40° C. The suspension was homogenized at 10,000×g with a ⅜″ MiniMicro workhead on a IART-A Silverson Laboratory Mixer for 10 min followed by 10 freeze-thaw cycles. Excess free VEGF was removed by centrifugation (4,000×g, 4° C. for 20 min), and replaced by 2 mL of sterile PBS, while free PDGF was kept in the formulation.

Liposome Characterization.

Liposomes were sized with a Beckmann Coulter Counter Multisizer 3 (Fullerton, Calif.). Zeta potentials were measured using Brookhaven Instruments Corporation ZetaP ALS and ZetaPlus software (Holtsville, N.Y.). Liposome drug concentrations were determined following disruption of the liposomes with octyl β-D-glucopyranoside (OGP, Sigma, St. Louis, Mo.). Lipid concentrations were determined by colorimetry using the Bartlett assay [35].

Preparation and Characterization of Hydrogels.

Several hydrogels were investigated to optimize cellular microenvironment for prolonged stability in vitro and/or in vivo, and to achieve desired release kinetics of the chosen growth factor. Their compositions are denoted as follows: hyaluronic acid, (HA, Mw=490 kDa and 1.4 MDa, Genzyme, Cambridge Mass.), carboxymethylcellulose (CMC; medium viscosity, Sigma, St. Louis, Mo.), dextran (DEX; 100 kDa, Sigma, St. Louis, Mo.). The polymers were modified with aldehyde modification, -CHO; or adipic hydrazide modification, -ADH to enable in situ crosslinking through formation of hydrazone bonds. Four hydrogels were tested: HA-CHO/HA-ADH, DEX-CHO/CMCADH, DEX-CHO/HA-ADH and CMC-CHO/CMC-ADH. Polymer modification was followed as previously described [22]. Briefly, for CHO modification, 1.5 g of DEX/HA/CMC, predissolved overnight in 150 mL of distilled water, was reacted with 802.1 mg of sodium periodate. After 2 h, 400 μL of ethylene glycol was added and the reaction was stirred for an additional 1 h. For ADH modification, 0.5 g of DEX/HA/CMC was dissolved in 100 mL of distilled water, and reacted with 1.5 g of ADH in the presence of 240 mg of 1-ethyl-3-[3-(dimethylamino)propyl]-carbodiimide (EDC, Sigma St. Louis, Mo.) and 240 mg of hydroxybenzotriazole (HOBt, Sigma St. Louis, Mo.) at pH 6.8 overnight at room temperature. The modified polymers were purified by dialysis for 3 days, followed by freeze drying. Hydrogels were produced using a double-barreled syringe (Baxter: Deerfield, Ill.). One barrel of the syringe contained 300 μl of ADH precursor (CMC or HA) solution in phosphate buffered saline (PBS), while the other was loaded with 300 μl of CHO precursor (CMC/HA/DEX) solution in PBS. The examined concentration of the various polymer precursor solutions are depicted in Table 2. Two hundred μL of liposomes (100 μL of each DSPC VEGF or DMPC PDG) or 100 μL when loaded together with 100 μL of hESCs (500,000−1×10⁶) were mixed in CHO precursor solution. In all cases the total volume was kept at 300 μl. The two solutions were merged by injection into a rubber mold or injected in vitro and/or in vivo, resulting in a solidified hydrogel. The diameters and the thicknesses of the prepared hydrogels were 1.2 cm and 3.5 mm, respectively.

Hydrogel Characterization.

The examined hydrogel compositions were characterized regarding their gelation time, swelling and stability in hESC media. Gelation time was measured by injecting ADH and CHO precursor solutions into a mold containing a stir bar (as previously described [23]). Stirring was set at 155 rpm using a Corning model PC-320 hot plate/stirrer. The gelation time was considered the time at which stir bar could no longer rotate inside the gels. Gelation time was measured five times for each hydrogel composition without additive, with liposomes or cells and with both liposomes and cells (n=5). Measurements were performed at 25 and 37° C. The time course of hydrogel swelling was measured gravimetrically as follows: The weight of the hydrogels was measured up to 5 weeks after immersion in hESC media (every day for the first week and then every 3 days thereafter). Hydrogel portions that remained intact were separated from degraded material and were transferred into fresh wells of solution before each measurement. The swelling ratio was calculated as the weight at a given time point divided by the initial weight of the hydrogel (following gelation). Human ESC-laden hydrogels were cultured in hESC media and degradation time course of the examined hydrogel compositions was followed daily and compared to hydrogels without cells

Liposome Formulation.

One mL of liposomes in solution was inserted into the lumen of a SpectraPor 1.1 Biotec Dispodialyzer (Spectrum Laboratories, Rancho Dominguez, Calif.) with a 50,000 MW cut-off. The dialysis bag was placed in a test tube with 12 mL cell culture medium and incubated at 37° C. on a tilt-table (Ames Aliquot, Miles). At predetermined intervals, the dialysis bag was transferred to a new test tube with fresh cell culture medium that was pre-warmed to 37° C.

Growth Factor Release from Hydrogels:

Hydrogels containing VEGF and PDGF growth factor either free in solution or encapsulated in liposomes (total of 200 μL) were weighed and placed in 12-well plates with inserts (for ease of gel transfer). Four mL of hESC culture medium was added to each well and the gels were incubated at 37° C. with constant rotation. Release medium was sampled (0.5 mL) at different time points and replaced with 4 mL of fresh cell

Culture Medium.

In addition, growth factor concentration within the hydrogels (not released to the media) was also measured to account for total growth factor concentration. This is particularly important for the PDGF concentration measurement since it has lower solubility in hydrophilic media, and will not diffuse easily in cell culture medium. At several time points (10 min, 1, 2, 4, 6, 24, 48, 96, and 120 hrs) hydrogels were crushed followed by centrifugation (4,000×g, 4° C. for 20 min) to separate the hydrogel debris and liposomes from the growth factors. VEGF and PDGF concentrations in the different samples were measured using an ELISA kit (R&D Systems, MN, USA).

Cell Culture.

Human ESCs (H9 clone) were grown on human foreskin fibroblasts (ATCC) in knockout media as previously described [36]. Induction of differentiation was performed by removing the cells from the feeder layer and transferring to petri dishes. This caused the formation of embryoid bodies (EBs) and induction of cell differentiation. EB formation was initiated in suspension in 15 cm plates and approximately 3,000,000 cells were generated in each plate [37]. Cells were incubated in the presence of EB media (80% knockout DMEM, 20% knockout serum, 1 mM glutamine, 0.1 mM beta mercaptoethanol and 1% non-essential amino acids). After 11 days, EBs were dissociated through trypsinization and CD34 positive cells were isolated using CD34 MicroBead KIT (Miltenyi Biotech, Auburn, Calif., USA) according to manufacturer instructions. Briefly, dissociated 11 days old EBs were labeled with the anti-CD34 antibody (QBEND/10, Miltenyi Biotec) conjugated with magnetic beads. The magnetically labeled cells were separated into CD34 positive and CD34 negative populations using a LS-MACS column (Miltenyi Biotec).

Transplantation into Ischemic Hindlimb Mouse Model.

Hindlimb ischemia was induced in an athymic mouse model (NCRNU, 20 g body weight; Taconic). The femoral artery was dissected and separated from the femoral vein and nerve at proximally near the groin and distally close to the knee. After the dissection, a strand of 7-0 polypropylene (Prolene) suture was placed underneath the proximal end of the femoral artery and the same was repeated at the distal location. Thus the femoral artery was excised from its proximal origin as a branch of the external iliac artery to the distal point where it bifurcates into the saphenous and popliteal arteries. Immediately after artery ligation and excision, hydrogel-forming compositions were injected on top of the ligated artery. Four experimental groups were examined as follows: CD34 positive cells, CD34 positive cell laden hydrogels, growth factor laden hydrogels, CD34 positive cells and growth factor laden hydrogels.

Immunohistochemistry

CD34 positive cell seeded hydrogels were cultured for 2 weeks, then embedded in Tissue Tek OCT (Sakura Finetek, Torrance Calif., USA) for cryosectioning. Sections were fixed with 4% paraformaldehyde and immuno-fluorescently labeled with anti-human CD31 (I:20) and a smooth muscle actin (a-SMA, 1:50) both obtained from R&D (Minneapolis, Minn., USA). Rhodamine conjugate secondary antibody was used for fluorescent visualization, followed by DAPI (4,6-diamidino-2-phenylindole) nuclear staining. Explants from animal experiments were harvested after 1, 4 and 6 weeks, fixed in 10% formalin and paraffin embedded. Immunohistochemical staining was carried out by using the Biocare Medical Universal HRP-DAB kit (Biocare Medical, Walnut Creek, Calif.) according to the manufacturer's instructions, with prior heat treatment at 90° C. for 20 min in ReVeal buffer (Biocare Medical) for epitope recovery. The primary antibodies were CD31 (1:20) and α-SMA (1:50).

In Vivo Ultrasound Imaging.

The ultrasound imaging was carried out using a Vevo 770 high-resolution microimaging system (VisualSonics Inc., Toronto, Canada) equipped with a broadband scanhead (RMV707B) centered at 30 MHz. The animals were anesthetized with 2% isoflurane in balanced air and restrained on the thermostated imaging platform in dorsal recumbency. The scanhead was secured directly above the hind limb. To allow positioning of the hind limb mid-section at the scanhead focal plane (12.7 mm from the probe face), a clear gel standoff was used for acoustic coupling. Two-dimensional axial B-mode scans of the hind limb were acquired over a 13 mm×13 mm field of view at a 50 Hz frame rate before and after intravenous administration of the MicroMarker (VisualSonics Inc., Toronto, Canada) contrast agent (1×10⁸ microbubbles in 100 μL saline) via a catheterized tail vein. Image processing was carried out using Matlab R2010a software package (MathWorks Inc.). Pseudo-color scale images revealing peak tissue contrast enhancement relative to the pre-injection baseline were overlaid on the grayscale anatomical scans.

### REFERENCES

[1] Bartholomew J R and Olin J W. (2006) Pathophysiology of peripheral arterial disease and risk factors for its development. Cleve Clin J Med; 73 Suppl 4: S8-14.
[2] Aranguren X L, Verfaillie C M and Luttun A. (2009) Emerging hurdles in stem cell therapy for peripheral vascular disease. J Mol Med; 87(1): 3-16.
[3] Collinson D J and Donnelly R. (2004) Therapeutic angiogenesis in peripheral arterial disease: can biotechnology produce an effective collateral circulation? Eur J Vasc Endovasc Surg; 28(I): 9-23.
[4] Khakoo A Y and Finkel T. (2005) Endothelial progenitor cells. Annu Rev Med; 56: 79-101.
[5] Asahara, Murohara, Sullivan, Silver, van der Zee, Li, et al. (1997) Isolation of putative progenitor endothelial cells for angiogenesis. Science; 275(5302): 964-7.
[6] Urbich C and Dimmeler S. (2004) Endothelial progenitor cells: characterization and role in vascular biology. Circ Res; 95(4): 343-53.
[7] Schmidt D, Breymann C, Weber A, Guenter C I, Neuenschwander S, Zund G, et al. (2004) Umbilical cord blood derived endothelial progenitor cells for tissue engineering of vascular grafts. Ann Thorac Surg; 78(6): 2094-8.
[8] Takahashi, Kalka, Masuda, Chen, Silver, Kearney, et al. (1999) Ischemia- and cytokine-induced mobilization of bone marrow-derived endothelial progenitor cells for neovascularization. Nat Med; 5(4): 434-8.
[9] Sharpe E E, 3rd, Teleron A A, Li 13, Price J, Sands M S, Alford K, et al. (2006) The origin and in vivo significance of murine and human culture-expanded endothelial progenitor cells. Am J Pathol; 168(5): 1710-21.
[10] Rafii S and Lyden D. (2003) Therapeutic stem and progenitor cell transplantation for organ vascularization and regeneration. Nat Med; 9(6): 702-12.
[11] Urbich C, Aicher A, Heeschen C, Dernbach E, Hofmann W K, Zeiher A M, et al. (2005) Soluble factors released by endothelial progenitor cells promote migration of endothelial cells and cardiac resident progenitor cells. J Mol Cell Cardiol; 39(5): 733-42.
[12] Schmidt D, Asmis L M, Odermatt B, Kelm J, Breymann C, Gossi M, et al. (2006) Engineered living blood vessels: functional endothelia generated from human umbilical cord-derived progenitors. Ann Thorac Surg; 82(4): 1465-71; discussion 1471.
[13] Patschan S A, Patschan D, Temme J, Korsten P, Wessels J T, Koziolek M, et al. (2011) Endothelial progenitor cells (EPc) in sepsis with acute renal dysfunction (ARD). Crit Care; 15(2): R94.
[14] Silva E A, Kim E S, Kong H J and Mooney D J. (2008) Material-based deployment enhances efficacy of endothelial progenitor cells. Proc Natl Acad Sci USA; 105(38): 14347-52.
[15] Chavakis E, Urbich C and Dimmeler S. (2008) Homing and engraftment of progenitor cells: a prerequisite for cell therapy. J Mol Cell Cardiol; 45(4): 514-22.
[16] Dornbach E, Randriamboavonjy V, Fleming I, Zeiher A M, Dimmeler Sand Urbich C. (2008) Impaired interaction of platelets with endothelial progenitor cells in patients with cardiovascular risk factors. Basic Res Cardiol; 103(6): 572
[17] Lovenberg S, Zoldan J, Basevitch Y and Langer R. (2007) Endothelial potential of human embryonic stem cells. Blood; 110(3): 806-14.
[18] Ferreira L S, Gerecht S, Shich H F, Watson N, Rupnick M A, Dallabrida S M, et al. (2007) Vascular progenitor cells isolated from human embryonic stem cells give rise to endothelial and smooth muscle like cells and form vascular networks in vivo. Circ Res; 101(3): 286-94.
[19] Kannan R Y, Salacinski H J, Sales K, Butler P and Seifalian A M. (2005) The roles of tissue engineering and vascularisation in the development of micro-vascular networks: a review. Biomaterials; 26(14): 1857-75.
[20] Richardson T P, Peters M C, Ennett A B and Mooney D J. (2001) Polymeric system for dual growth factor delivery. Nat Biotechnol; 19(11): 1029-34.
[21] Chen R R, Silva E A, Yuen W W and Mooney D J. (2007) Spatio-temporal VEGF and PDGF delivery patterns in blood vessel formation and maturation. Pharm Res; 24(2): 258-64.
[22] Ito T, Yeo Y, Highley C B, Bellas E and Kohane D S. (2007) Dextran-based in situ cross-linked injectable hydrogels to prevent peritoneal adhesions. Biomaterials; 28(23): 3418-26.
[23] Yeo Y, Highley C B, Bellas E, Ito T, Marini R, Langer R, et al. (2006) In situ cross-linkable hyaluronic acid hydrogels prevent post-operative abdominal adhesions in a rabbit model. Biomaterials; 27(27): 4698-705.
[24] Gerecht S, Burdick J A, Ferreira L S, Townsend S A, Langer R. and Vunjak-Novakovic G. (2007) Hyaluronic acid hydrogel for controlled self-renewal and differentiation of human embryonic stem cells. Proc Natl Acad Sci USA; 104(27): 11298-303.
[25] Canneliet P. (2000) Mechanisms of angiogenesis and arteriogenesis. Nat Med. 2000 April; 6(4):389-95.
[26] Coultas L, Chawengsaksophak K and Rossant J. (2005) Endothelial cells and VEGF in vascular development. Nature 438(7070): 937-945.
[27] Eichmann A, Yuan L, Moyon D, Lenoble F, Pardanaud Land Breant C. (2005) Vascular development: from precursor cells to branched arterial and venous networks. Int J Dev Biol; 49(2-3): 259-67.
[28] Ferrara N, Gerber H-P and LeCouter J. (2003) The biology of VEGF and its receptors. Nat Med. 2003 June; 9(6):669-76.
[29] Jain R K. (2003) Molecular regulation of vessel maturation. Nat Med. 2003 June; 9(6):685-93.
[30] Cho S W, Moon S H, Lee S H, Kang S W, Kim J, Lim J M, et al. (2007) Improvement of postnatal neovascularization by human embryonic stem cell derived endothelial-like cell transplantation in a mouse model of hindlimb ischemia. Circulation; 116(21): 2409-19.
[31] Sone M, Hoh H, Yamahara K, Yamashita J K, Yurugi-Kobayashi T, Nonoguchi A, et al. (2007) Pathway for differentiation of human embryonic stem cells to vascular cell components and their potential for vascular regeneration. Arterioscler Thromb Vasc Biol; 27(I0): 2127-34.
[32] Yamahara K, Sone M, Itoh H, Yamashita J K, Yurugi-Kobayashi T, Homma K, et al. (2008) Augmentation of neovascularization in hindlimb ischemia by combined transplantation of human embryonic stem cells-derived endothelial and mural cells. PLoS One; 3(2): e1666.
[33] Lu S J, Feng Q, Caballero S, Chen Y, Moore M A, Grant M B, et al. (2007) Generation of functional hemangioblasts from human embryonic stem cells. Nat Methods; 4(6): 501-9.
[34] Szoka F, Jr. and Papahadjopoulos D. (1980) Comparative properties and methods of preparation of lipid vesicles (liposomes). Annu Rev Biophys Bioeng; 9: 467-508.
[35] Bartlett G R. (1959) Phosphorus assay in column chromatography. J Biol Chem; 234(3): 466-8.
[36] Amit M, Margulets V, Segev 11, Shariki K, Laevsky I, Coleman R, et al. (2003) Human feeder layers for human embryonic stem cells. Biol Reprod; 68(6): 2150-6.
[37] Zoldan J and Levenberg S. (2006) Engineering three-dimensional tissue structures using stem cells. Methods Enzymol; 420: 381-91.

All publications, patents, patent applications, and sequence database entries mentioned herein are hereby incorporated by reference in their entirety as if each individual publication or patent was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group or can be explicitly disclaimed. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange and any individual value within the given range, wherein the endpoints of the subrange are expressed to the same degree of accuracy as the tenth of the unit of the lower limit of the range, and the individual values can assume any value within the range to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A hydrogel comprising
(a) a population of stem or progenitor cells that differentiate into a desired cell type in response to a growth factor;
(b) the growth factor of (a) in a controlled-release form; and
(c) a hydrogel scaffold encapsulating the cells of (a) and the controlled-release form of (b).

2. The hydrogel of claim 1, wherein the hydrogel comprises a plurality of growth factors.

3. The hydrogel of claim 2, wherein at least two growth factors are in different controlled-release forms.

4. The hydrogel of claim 3, wherein the different controlled-release forms exhibit different release kinetics.

5. The hydrogel of claim 3, wherein the different controlled-release forms exhibit different rates of release.

6. The hydrogel of claim 1, wherein the controlled-release form is a liposome-encapsulated form.

7. The hydrogel of claim 6, wherein the liposomes in which the growth factors are encapsulated are selected from the group consisting of DMPC liposomes (high rate of release) and DSPC liposomes (low rate of release).

8. The hydrogel of claim 1, wherein the cells differentiate into cells that form blood vessels in response to the growth factor.

9. The hydrogel of claim 1, wherein the population of cells comprises endothelial progenitor cells.

10. The hydrogel of claim 1, wherein the hydrogel comprises VEGF in a controlled-release form exhibiting a high rate of release and PDGF in a controlled-release form exhibiting a low rate of release.

11. The hydrogel of claim 1, wherein the hydrogel scaffold comprises a polysaccharide.

12. The hydrogel of claim 11, wherein the polysaccharide of the hydrogel scaffold is selected from the group consisting of carboxymethylcellulose (CMC), hyaluronic acid (HA), and dextran (DEX).

13. The hydrogel of claim 1, wherein the hydrogel scaffold comprises a plurality of polysaccharide molecules that are covalently bound to each other via hydrazone bonds.

14. The hydrogel of claim 1, wherein the average pore size of the hydrogel is smaller than the average diameter of the cells of (a) and/or than the average diameter of the controlled-release form of (b).

15. A composition comprising
(a) a growth factor in a controlled-release form;
(b) a polymer comprising a first reactive moiety; and
(c) a polymer comprising a second reactive moiety that forms a covalent bond with the first reactive moiety under physiological conditions, thus forming a hydrogel.

16-32. (canceled)

33. A method comprising,
administering the hydrogel of claim 1 to a subject in need thereof.

34-35. (canceled)

36. A method, comprising
providing
(a) a growth factor in a controlled-release form;
(b) a polymer comprising a first reactive moiety; and
(c) a polymer comprising a second reactive moiety that forms a covalent bond with the first reactive moiety under physiological conditions; and
contacting the polymer of (b) with the polymer of (c) in the presence of the growth factor of (a), thus forming a hydrogel encapsulating the growth factor of (a).

37-56. (canceled)

57. A kit comprising
(a) a polymer comprising a first reactive moiety; and
(b) a polymer comprising a second reactive moiety, wherein the second reactive moiety forms a covalent bond with the first reactive moiety under physiological conditions, thus forming a hydrogel comprising the polymer of (a) covalently bound to the polymer of (b).

58-65. (canceled)
